(19) United States
(12) Patent Application Publication
(10) Pub. No.: US 2017/0258843 A1
(43) Pub. Date: Sep. 14, 2017

(54) STEM CELL MEDIATED NEUROREGENERATION AND NEUROPROTECTION

(71) Applicant: AngioStem, Inc., Phoenix, AZ (US)

(72) Inventors: Thomas Ichim, San Diego, CA (US); Amit Patel, Salt Lake City, UT (US)

(21) Appl. No.: 15/458,847

(22) Filed: Mar. 14, 2017

Related U.S. Application Data
(60) Provisional application No. 62/308,199, filed on Mar. 14, 2016.

(51) Int. Cl. A61K 35/28; A61K 35/50

(52) U.S. Cl. CPC A61K 35/28; A61K 35/50

(57) ABSTRACT

Disclosed are means of inducing neuroregeneration and/or neuroprotection in patients with damage to the nervous system. In one embodiment, placenta derived CD34 positive cells are administered to a patient suffering from a neurological injury, said cells administered alone, or in combination with endothelial progenitor cells that are derived from placental sources. In one embodiment cells are manipulated to decrease immunogenicity by means of gene-editing or RNA interference inducing means. In another embodiment, neuroprotection and/or neuroregeneration is achieved by administration of exosomes derived from placental stem cells.

### CROSS-REFERENCE TO RELATED APPLICATIONS

The present invention claims priority to U.S. Provisional Application No. 62/308,199, filed Mar. 14, 2016, which is hereby incorporated in its entirety including all tables, figures, and claims

### FIELD OF INVENTION

The invention belongs to the field of cancer therapeutics, more specifically the invention belongs to the field of enabling dose escalation of chemo, radiation or immunotherapy, to selectively protect non-malignant tissue from effects of said therapy. In another embodiment the invention provide use of regenerative cells to rebuild non-neoplastic tissue

### BACKGROUND OF THE INVENTION

Radiotherapy, the clinical application of various types of ionizing radiation, is a major therapeutic intervention in modern cancer therapy in addition to surgery and chemotherapy. This is corroborated by the fact that more than 60% of all cancer patients receive one form of radiotherapy [1]. Cranial irradiation is commonly used for the treatment of neoplasms involving the central nervous system. Radiotherapy can have negative sequelae of acute neurocognitive deficits, especially in the pediatric population [2]. The pathogenesis of radiation-induced neurocognitive deficits involves apoptosis of neuroproliferative cells in the subgranular zone of the hippocampus, a region in the brain vital for learning and memory [3, 4]. Several studies have demonstrated a steep, long-term decline in subgranular neurogenesis in the dentate gyrus following radiation exposure [5] and direct irradiation of the hippocampus has been shown to result in pronounced cognitive deficits [6]. The cognitive deficits following hippocampal irradiation include deficits of learning, memory, and ability for spatial processing [7]. To date, means of selectively protecting non-malignant tissue, especially regenerative tissue, from neurodegenerative effects of radiotherapy, and/or chemotherapy, are non-existent. Small molecule antioxidants have been shown to possess some efficacy in animal models, however, clinical data is lacking [8].

### DESCRIPTION OF THE INVENTION

The invention provides means of selectively protect healthy tissue from radiation or chemotherapy by administration of regenerative cells. In one embodiment the invention provides cells that selectively home to signals generated by non-malignant brain tissue in response to irradiation or chemotherapy.

The invention provides compositions of matter, protocols and uses of regenerative cells aimed at reducing and/or ameliorating neurodegenerative effects of brain cancer targeted therapeutics. In one embodiment the invention teaches the use of self-renewing cells with ability to provide anti-inflammatory and/or neuroprotective activities which mediate selective effects on non-malignant tissue, while allowing for chemotherapy and/or radiotherapy to target malignant tissue. In one specific embodiment, mesenchymal lineage stem cells art utilized to selectively provide super oxide dismutase to non-neoplastic tissue, thus protecting endogenous non-malignant stem cells, for example in the dendtate gyrus and subventricular zone of the brain, while allowing for death, mitotic inactivation and autophagy of neoplastic brain cells in response to radiation and/or chemotherapy. In another embodiment, self-renewing cells are utilized post chemotherapy and/or radiation therapy to allow for amelioration of neurocognitive effects of said chemotherapy and/or radiation therapy.

It is known in the art that tumor cells lose specific physiological functions that are found in non-malignant cells in order to focus energy expenditure and cellular activities on proliferation, apoptosis resistance, and metastasis. Examples of such “focusing of resources” can be seen in the case of proteasomes, in which tumors lose several proteasomes found in non-malignant cells, thus reducing redundancy of protein degradation activity. Given activity, proteasome inhibitors such as bortezomib, have been shown to selectively kill cancer cells, which have lost redundancy, whereas healthy cells do not succumb to proteasome inhibition due to existing redundancy of protein degradation pathways [9]. Similarly, the current invention is based on the unexpected finding that tumor cells possess a reduced ability to evoke stem cell chemotactic responses after injury as compared to non-malignant brain tissue. In one embodiment the invention teaches the use of various stem cells for protection, treatment, and restoration of neurological function subsequent to chemotherapy and/or radiation therapy of brain tumors.

“Treat” or “treatment” means improving the rate of accelerating healing or completely healing a pathology. In the case of wound healing, methods for measuring the rate of wound healing are known in the art and include, for example, observing increased epithelialization and/or granulation tissue formation, or lessening of the wound diameter and/or depth. Increased epithelialization can be measured by methods known in the art such as by, for example, the appearance of new epithelium at the wound edges and/or new epithelial islands migrating upward from hair follicles and sweat glands.

“Angiogenesis” means any alteration of an existing vascular bed or the formation of new vasculature which benefits tissue perfusion. This includes the formation of new vessels by sprouting of endothelial cells from existing blood vessels or the remodeling of existing vessels to alter size, maturity, direction or flow properties to improve blood perfusion of tissues. As used herein the terms, “angiogenesis,” “revascularization,” “increased collateral circulation,” and “regeneration of blood vessels” are considered as synonymous.

“Chronic wound” means a wound that has not completely closed in twelve weeks since the occurrence of the wound in a patient having a condition, disease or therapy associated with defective healing. Conditions, diseases or therapies associated with defective healing include, for example, diabetes, arterial insufficiency, venous insufficiency, chronic steroid use, cancer chemotherapy, radiotherapy, radiation exposure, and malnutrition. A chronic wound includes defects resulting in inflammatory excess (e.g., excessive production of Interleukin-6 (IL-6), tumor necrosis factor-alpha (TNF-.alpha.), and MMPs), a deficiency of important growth factors needed for proper healing, bacterial overgrowth and senescence of fibroblasts. A chronic wound has an epithelial layer that fails to cover the entire surface of the wound and is subject to bacterial colonization.

“Therapeutically effective amount” means the amount of cells, conditioned media or exosomes that, when administered to a mammal for treating a chronic wound, or angiogenic insufficiency is sufficient to effect such treatment. The “therapeutically effective amount” may vary depending on the size of the wound, and the age, weight, physical condition and responsiveness of the mammal to be treated.

DNA “coding sequence” refers to a double-stranded DNA sequence that encodes a polypeptide and can be transcribed and translated into a polypeptide in a cell in vitro or in vivo when placed under the control of suitable regulatory sequences. “Suitable regulatory sequences” refers to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and even synthetic DNA sequences. If the coding sequence is intended for expression in an eukaryotic cell, a polyadenylation signal and transcription termination sequence will usually be located 3′ to the coding sequence.

“Open reading frame” is abbreviated ORF and refers to a length of nucleic acid sequence, either DNA, cDNA or RNA, that comprises a translation start signal or initiation codon, such as an ATG or AUG, and a termination codon and can be potentially translated into a polypeptide sequence.

“head-to-head” is used herein to describe the orientation of two polynucleotide sequences in relation to each other. Two polynucleotides are positioned in a head-to-head orientation when the 5′ end of the coding strand of one polynucleotide is adjacent to the 5′ end of the coding strand of the other polynucleotide, whereby the direction of transcription of each polynucleotide proceeds away from the 5′ end of the other polynucleotide. The term “head-to-head” may be abbreviated (5′)-to-(5′) and may also be indicated by the symbols (.rarw. .fwdarw.) or (3′.rarw.5′5′.fwdarw.3′).

“tail-to-tail” is used herein to describe the orientation of two polynucleotide sequences in relation to each other. Two polynucleotides are positioned in a tail-to-tail orientation when the 3′ end of the coding strand of one polynucleotide is adjacent to the 3′ end of the coding strand of the other polynucleotide, whereby the direction of transcription of each polynucleotide proceeds toward the other polynucleotide. The term “tail-to-tail” may be abbreviated (3′)-to-(3′) and may also be indicated by the symbols (.fwdarw. .rarw.) or (5′.fwdarw.3′3.rarw.5′).

“head-to-tail” is used herein to describe the orientation of two polynucleotide sequences in relation to each other. Two polynucleotides are positioned in a head-to-tail orientation when the 5′ end of the coding strand of one polynucleotide is adjacent to the 3′ end of the coding strand of the other polynucleotide, whereby the direction of transcription of each polynucleotide proceeds in the same direction as that of the other polynucleotide. The term “head-to-tail” may be abbreviated (5′)-to-(3′) and may also be indicated by the symbols (.fwdarw. .fwdarw.) or (5′.fwdarw.3′5′.fwdarw.3′).

“downstream” refers to a nucleotide sequence that is located 3′ to a reference nucleotide sequence. In particular, downstream nucleotide sequences generally relate to sequences that follow the starting point of transcription. For example, the translation initiation codon of a gene is located downstream of the start site of transcription.

“upstream” refers to a nucleotide sequence that is located 5′ to a reference nucleotide sequence. In particular, upstream nucleotide sequences generally relate to sequences that are located on the 5′ side of a coding sequence or starting point of transcription. For example, most promoters are located upstream of the start site of transcription.

“restriction endonuclease” and “restriction enzyme” are used interchangeably and refer to an enzyme that binds and cuts within a specific nucleotide sequence within double stranded DNA.

“Therapeutic agent” means to have “therapeutic efficacy” in modulating angiogenesis and/or wound healing/and/or treatment of a pathology through augmentation of a desired therapeutic effect. In a specific embodiment the amount of the therapeutic is said to be a “angiogenic modulatory amount”, if administration of that amount of the therapeutic is sufficient to cause a significant modulation (i.e., increase or decrease) in angiogenic activity when administered to a subject (e.g., an animal model or human patient) needing modulation of angiogenesis.

“Growth factor” can be a naturally occurring, endogenous or exogenous protein, or recombinant protein, capable of stimulating cellular proliferation and/or cellular differentiation and cellular migration.

“About” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise stated, the term ‘about’ means within an acceptable error range for the particular value.

“Pharmaceutically acceptable” refers to a natural or synthetic substance means that the substance has an acceptable toxic effect in view of its much greater beneficial effect, while the related the term, “physiologically acceptable,” means the substance has relatively low toxicity.

“expression vector” refers to a vector, plasmid or vehicle designed to enable the expression of an inserted nucleic acid sequence following transformation into the host. The cloned gene, i.e., the inserted nucleic acid sequence, is usually placed under the control of control elements such as a promoter, a minimal promoter, an enhancer, or the like. Initiation control regions or promoters, which are useful to drive expression of a nucleic acid in the desired host cell are numerous and familiar to those skilled in the art. Virtually any promoter capable of driving expression of these genes can be used in an expression vector, including but not limited to, viral promoters, bacterial promoters, animal promoters, mammalian promoters, synthetic promoters, constitutive promoters, tissue specific promoters, pathogenesis or disease related promoters, developmental specific promoters, inducible promoters, light regulated promoters; CYC1, HIS3, GAL1, GAL4, GAL10, ADH1, PGK, PHO5, GAPDH, ADC1, TRP1, URA3, LEU2, ENO, TPI, alkaline phosphatase promoters (useful for expression in Saccharomyces); AOX1 promoter (useful for expression in Pichia); .beta.-lactamase, lac, ara, tet, trp,1P.sub.L, 1P.sub.R, T7, tac, and trc promoters (useful for expression in Escherichia coli); light regulated-,seed specific-, pollen specific-, ovary specific-, cauliflower mosaic virus 35S, CMV 35S minimal, cassava vein mosaic virus (CsVMV), chlorophyll a/b binding protein, ribulose 1,5-bisphosphate carboxylase, shoot-specific, root specific, chitinase, stress inducible, rice tungro bacilliform virus, plant super-promoter, potato leucine aminopeptidase, nitrate reductase, mannopine synthase, nopaline synthase, ubiquitin, zein protein, and anthocyanin promoters (useful for expression in plant cells); animal and mammalian promoters known in the art including, but are not limited to, the SV40 early (SV40e) promoter region, the promoter contained in the 3′ long terminal repeat (LTR) of Rous sarcoma virus (RSV), the promoters of the E1A or major late promoter (MLP) genes of adenoviruses (Ad), the cytomegalovirus (CMV) early promoter, the herpes simplex virus (HSV) thymidine kinase (TK) promoter, a baculovirus 1E1 promoter, an elongation factor 1 alpha (EF1) promoter, a phosphoglycerate kinase (PGK) promoter, a ubiquitin (Ubc) promoter, an albumin promoter, the regulatory sequences of the mouse metallothionein-L promoter and transcriptional control regions, the ubiquitous promoters (HPRT, vimentin, .alpha.-actin, tubulin and the like), the promoters of the intermediate filaments (desmin, neurofilaments, keratin, GFAP, and the like), the promoters of therapeutic genes (of the MDR, CFTR or factor VIII type, and the like), pathogenesis or disease related-promoters, and promoters that exhibit tissue specificity and have been utilized in transgenic animals, such as the elastase I gene control region which is active in pancreatic acinar cells; insulin gene control region active in pancreatic beta cells, immunoglobulin gene control region active in lymphoid cells, mouse mammary tumor virus control region active in testicular, breast, lymphoid and mast cells; albumin gene, Apo AI and Apo All control regions active in liver, alpha-fetoprotein gene control region active in liver, alpha 1-antitrypsin gene control region active in the liver, beta-globin gene control region active in myeloid cells, myelin basic protein gene control region active in oligodendrocyte cells in the brain, myosin light chain-2 gene control region active in skeletal muscle, and gonadotropic releasing hormone gene control region active in the hypothalamus, pyruvate kinase promoter, villin promoter, promoter of the fatty acid binding intestinal protein, promoter of the smooth muscle cell .alpha.-actin, and the like. In addition, these expression sequences may be modified by addition of enhancer or regulatory sequences and the like.

“Growth Factor” means any protein, polypeptide, variant or portion thereof that is capable of, directly or indirectly, including endothelial cell growth. Such proteins include, for example, acidic and basic fibroblast growth factors (aFGF) (GenBank Accession No. NP.sub.--149127) and bFGF (GenBank Accession No. AAA52448), vascular endothelial growth factor (VEGF) (GenBank Accession No. AAA35789 or NP.sub.--001020539), epidermal growth factor (EGF) (GenBank Accession No. NP.sub.--001954), transforming growth factor .alpha. (TGF-.alpha.) (GenBank Accession No. NP.sub.--003227) and transforming growth factor .beta. (TFG-.beta.) (GenBank Accession No. 1109243A), platelet-derived endothelial cell growth factor (PD-ECGF) (GenBank Accession No. NP.sub.--001944), platelet-derived growth factor (PDGF) (GenBank Accession No. 1109245A), tumor necrosis factor .alpha. (TN-.alpha.) (GenBank Accession No. CAA26669), hepatocyte growth factor (HGF) (GenBank Accession No. BAA14348), insulin like growth factor (IGF) (GenBank Accession No. P08833), erythropoietin (GenBank Accession No. P01588), colony stimulating factor (CSF), macrophage-CSF (M-CSF) (GenBank Accession No. AAB59527), granulocyte/macrophage CSF (GM-CSF) (GenBank Accession No. NP.sub.--000749), monocyte chemotactic protein-1 (GenBank Accession No. P13500) and nitric oxide synthase (NOS) (GenBank Accession No. AAA36365). See, Klagsbrun, et al., Annu. Rev. Physiol., 53:217-239 (1991); Folkman, et al., J. Biol. Chem., 267:10931-10934 (1992) and Symes, et al., Current Opinion in Lipidology, 5:305-312 (1994). Variants or fragments of a mitogen may be used as long as they induce or promote endothelial cell or endothelial progenitor cell growth. Preferably, the endothelial cell mitogen contains a secretory signal sequence that facilitates secretion of the protein. Proteins having native signal sequences, e.g., VEGF, are preferred. Proteins that do not have native signal sequences, e.g., bFGF, can be modified to contain such sequences using routine genetic manipulation techniques. See, Nabel et al., Nature, 362:844 (1993).

“Mesenchymal stem cell” or “MSC” refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. As used herein, “mesenchymal stromal cell” or “MSC” can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. As used herein, “mesenchymal stromal cell” or “MSC” includes cells that are CD34 positive upon initial isolation from tissue but are similar to cells described about phenotypically and functionally. As used herein, “MSC” includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. As used herein, “mesenchymal stromal cell” or “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, Stempeuce1CLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

“vector” refers to any vehicle for the cloning of and/or transfer of a nucleic acid into a host cell. A vector may be a replicon to which another DNA segment may be attached so as to bring about the replication of the attached segment. A “replicon” refers to any genetic element (e.g., plasmid, phage, cosmid, chromosome, virus) that functions as an autonomous unit of DNA replication in vivo, i.e., capable of replication under its own control. The term “vector” includes both viral and nonviral vehicles for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacteriophages such as lambda derivatives, or plasmids such as pBR322 or pUC plasmid derivatives, or the Bluescript vector. Another example of vectors that are useful in the invention is the UltraVector.™. Production System (Intrexon Corp., Blacksburg, Va.) as described in WO 2007/038276. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker. Viral vectors, and particularly retroviral vectors, have been used in a wide variety of gene delivery applications in cells, as well as living animal subjects. Viral vectors that can be used include, but are not limited to, retrovirus, adeno-associated virus, pox, baculovirus, vaccinia, herpes simplex, Epstein-Barr, adenovirus, geminivirus, and caulimovirus vectors. Non-viral vectors include plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2 [symbol] 10⁷ cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1 [symbol] 10⁶ cells per ml in 175 cm² tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1 [symbol] 10⁶ per 175 cm². Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

In some embodiments of the invention MSC are transferred to possess enhanced neuromodulatory and neuroprotective properties. Said transfection may be accomplished by use of lentiviral vectors, said means to perform lentiviral mediated transfection are well-known in the art and discussed in the following references [10-16]. Some specific examples of lentiviral based transfection of genes into MSC include transfection of SDF-1 to promote stem cell homing, particularly hematopoietic stem cells [17], GDNF to treat Parkinson's in an animal model [18], HGF to accelerate remyelination in a brain injury model [19], akt to protect against pathological cardiac remodeling and cardiomyocyte death [20], TRAIL to induce apoptosis of tumor cells [21-24], PGE-1 synthase for cardioprotection [25], NUR77 to enhance migration [26], BDNF to reduce ocular nerve damage in response to hypertension [27], HIF-1 alpha to stimulate osteogenesis [28], dominant negative CCL2 to reduce lung fibrosis [29], interferon beta to reduce tumor progression [30], HLA-G to enhance immune suppressive activity [31], hTERT to induce differentiation along the hepatocyte lineage [32], cytosine deaminase [33], OCT-4 to reduce senescence [34, 35], BAMBI to reduce TGF expression and protumor effects [36], HO-1 for radioprotection [37], LIGHT to induce antitumor activity [38], miR-126 to enhance angiogenesis [39, 40], bc1-2 to induce generation of nucleus pulposus cells [41], telomerase to induce neurogenesis [42], CXCR4 to accelerate hematopoietic recovery [43] and reduce unwanted immunity [44], wntll to promote regenerative cytokine production [45], and the HGF antagonist NK4 to reduce cancer [46].

Cell cultures are tested for sterility weekly, endotoxin by limulus amebocyte lysate test, and mycoplasma by DNA-fluorochrome stain.

In order to determine the quality of MSC cultures, flow cytometry is performed on all cultures for surface expression of SH-2, SH-3, SH-4 MSC markers and lack of contaminating CD14-and CD-45 positive cells. Cells were detached with 0.05% trypsin-EDTA , washed with DPBS +2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody . Confluent MSC in 175 cm² flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington D.C., USA). MSCs harvested from each 10-flask set were stored for up to 4 h at 4° C. and combined at the end of the harvest. A total of 2-10 [symbol] 10⁶ MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, lll., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In one embodiment of the invention MSC are transfected with anti-apoptotic proteins to enhance in vivo longevity. The present invention includes a method of using MSC that have been cultured under conditions to express increased amounts of at least one anti-apoptotic protein as a therapy to inhibit or prevent apoptosis. In one embodiment, the MSC which are used as a therapy to inhibit or prevent apoptosis have been contacted with an apoptotic cell. The invention is based on the discovery that MSC that have been contacted with an apoptotic cell express high levels of anti-apoptotic molecules. In some instances, the MSC that have been contacted with an apoptotic cell secrete high levels of at least one anti-apoptotic protein, including but not limited to, STC-1, BCL-2, XIAP, Survivin, and Bc1-2XL. Methods of transfecting antiapoptotic genes into MSC have been previously described which can be applied to the current invention, said antiapoptotic genes that can be utilized for practice of the invention, in a nonlimiting way, include GATA-4 [47], FGF-2 [48], bc1-2 [41, 49], and HO-1[50]. Based upon the disclosure provided herein, MSC can be obtained from any source. The MSC may be autologous with respect to the recipient (obtained from the same host) or allogeneic with respect to the recipient. In addition, the MSC may be xenogeneic to the recipient (obtained from an animal of a different species). In one embodiment of the invention MSC are pretreated with agents to induce expression of antiapoptotic genes, one example is pretreatment with exendin-4 as previously described [51]. In a further non-limiting embodiment, MSC used in the present invention can be isolated, from the bone marrow of any species of mammal, including but not limited to, human, mouse, rat, ape, gibbon, bovine. In a non-limiting embodiment, the MSC are isolated from a human, a mouse, or a rat. In another non-limiting embodiment, the MSC are isolated from a human.

Based upon the present disclosure, MSC can be isolated and expanded in culture in vitro to obtain sufficient numbers of cells for use in the methods described herein provided that the MSC are cultured in a manner that promotes contact with a tumor endothelial cell. For example, MSC can be isolated from human bone marrow and cultured in complete medium (DMEM low glucose containing 4 mM L-glutamine, 10% FBS, and 1% penicillin/streptomycin) in hanging drops or on non-adherent dishes. The invention, however, should in no way be construed to be limited to any one method of isolating and/or to any culturing medium. Rather, any method of isolating and any culturing medium should be construed to be included in the present invention provided that the MSC are cultured in a manner that provides MSC to express increased amounts of at least one anti-apoptotic protein. Culture conditions for growth of clinical grade MSC have been described in the literature and are incorporated by reference [52-85].

Endothelial progenitor cells are a population of rare cells that circulate in the blood with the ability to differentiate into endothelial cells. Without limiting the present invention to any one theory or mode of action, endothelial progenitor cells were first believed to be angioblasts, these being stem cells that form blood vessels during embryogenesis. While embryonic angioblasts have been known to exist for many years, adult endothelial progenitor cells were first believed to be characterized in the 1990s after Asahara and colleagues published that a purified population of CD34.sup.+ cells isolated from the blood of adult mice could purportedly differentiate into endothelial cells in vitro. Accordingly, reference to “endothelial progenitor cell” should be understood as a reference to any cell that exhibits the potentiality to develop to a cell exhibiting one or more of the functional or structural characteristics that are exhibited by an endothelial cell. Still without limiting the present invention in any way, reference to “endothelial cell” should be understood as a reference to the squamous epithelial cells that line the blood vessels, lymphatics or other serous cavities such as fluid-filled cavities. The phrase “endothelial cells” should also be understood as a reference to cells that exhibit one or more of the morphology, phenotype and/or functional activity of endothelial cells and is also a reference to mutants or variants thereof. Said endothelial cells may be at any differentiative stage of development subsequent to the endothelial progenitor cell stage. “Variants” include, but are not limited to, cells exhibiting some but not all of the morphological or phenotypic features or functional activities of endothelial cells. “Mutants” include, but are not limited to, endothelial cells which are genetically modified, such as endothelial cells derived from endothelial progenitor cells which are genetically modified subsequently to isolation by the method of the present invention but prior to undergoing directed differentiation along the endothelial cell lineage. Preferably, the subject endothelial cells are blood vessel endothelial cells (i.e., endothelial cells which form blood vessels) or are an immature form of endothelial cells which would proliferate and differentiate to form a blood vessel but which are nevertheless more mature than an endothelial progenitor cell.

According to this embodiment, there is provided a method of isolating mammalian endothelial progenitor cells said method comprising the steps of: [0145] (i) isolating a mammalian cellular population; [0146] (ii) enriching for a subpopulation of the cells of step (i), which subpopulation expresses a CD45.sup.− phenotypic profile; [0147] (iii) enriching for a subpopulation of the CD45.sup.− cells derived from step (ii) which express a CD34.sup.+ phenotypic profile; and [0148] (iv) isolating the subpopulation of CD34.sup.+ cells derived from step (iii) which express a CD31.sup.lo/− phenotypic profile, to thereby isolate the endothelial progenitor cells. and wherein said endothelial progenitor cell is capable of differentiating to a vascular endothelial cell.

In one embodiment steps (ii) to (iv) are performed sequentially. Without limiting the present invention to any one theory or mode of action, in addition to the bone marrow stroma comprising hematopoietic stem cells it is also known to comprise other non-hematopoietic stem cells, termed mesenchymal stem cells, the latter cell type being mesodermally derived and also being capable of both self renewal and differentiation, inter alia, to bone, cartilage, muscle, tendon, ligament, stroma, marrow, fat, neurons and astrocytes. Mesenchymal stem cells are also similar to hematopoietic stem cells in that they are very rare, existing at an estimated frequency of 1 in 100,000 bone marrow cells. Mature, fully differentiated mesenchymal-derived cells are the result of a step-wise maturation process termed mesogenesis. In addition to their localization to the bone marrow, mesenchymal stem cells are also found in a variety of other tissues including, but not limited to fat, bone, dental pulp and uterus.

Still without limiting the present invention in any way, the mature cell types to which mesenchymal stem cells give rise can contribute to the formation of the connective tissue of the organ in issue. “Connective tissue” is a generalized term for mesodermally derived tissue which may be more or less specialized. For example, cartilage and bone are forms of specialized connective tissue, as is blood. Other forms of less specialized connective tissue includes the tissues which are rich in extracellular matrix and surround other more highly ordered tissues and organs. Connective tissue therefore comprises many cell types that exhibit a variety of functions.

Reference to a “mesenchymal stem cell” should therefore be understood as a reference to any cell which exhibits the potentiality to develop to a cell exhibiting one or more of the functional or structural characteristics which are exhibited by a mesenchymal or mesenchymal-derived cell but not a non-mesenchymal-derived cell such as an endodermal or mesodermal derived cell type. Mesenchymal stem cells are also alternatively known as “stromal stem cells”, “fetal stem cells”, “adult stem cells”, “adipose derived stem cells”, “lipoaspirate derived stem cells” and “post natal stem cells”. To this end, reference to “mesenchymal-derived cell” should be understood as a reference to cell types that are more differentiated than a pluripotent mesenchymal cell and which have arisen from a mesenchymal stem cell. These cells will correspond to cells of the tissues to which mesenchymal cells are known to give rise and which have been detailed hereinbefore. For example, the subject mesenchymal-derived cell may be a cell which is irreversibly committed to differentiating along a particular cell lineage, such as a myocytic precursor cell or adipocytic precursor cell, or it may correspond to a partially or terminally differentiated form of a specific cellular subtype of one of these lineages. Accordingly, mesenchymal stem cells exhibit the ability to differentiate to a cell type of one or more of the mesenchymal lineages under appropriate conditions

The mesenchymal stem cells that are identified in accordance with the method of the invention are defined as cells that are not terminally differentiated. Accordingly, although it is a preferred embodiment that the subject cells are capable of differentiating along any mesenchymal lineage or even some non-mesenchymal lineages such as neuroectodermal cells (i.e., pluripotent mesenchymal cells), they may also correspond to cells that are capable of differentiating along just some of the mesenchymal lineages.

By “progenitor cell” and “stem cell” is meant that the cell is not fully differentiated but requires further differentiation to achieve maturation. Such cells also typically exhibit a higher degree of proliferation capacity than is exhibited by a fully differentiated cell. This proliferation capacity is also referred to as self-renewal capacity. Progenitor cells are capable of forming bigger colonies (i.e. they undergo a high level of proliferation) while more differentiated cells form smaller colonies. Fully differentiated cells do not form colonies. Progenitor cells and stem cells are also sometimes referred to as “precursor” cells “multipotent” cells, or “pluripotent” cells (although the latter term is generally reserved for cells which exhibit extensive potentiality). In this regard, stem cells are also generally regarded as a cell which exhibits wider potentiality than progenitor cells, as is exemplified herein where a mesenchymal stem cell has the potential to differentiate into a wider range of somatic cell types than an endothelial progenitor cell. It should be understood that the endothelial progenitor cell of the present invention may be monopotent or multipotent. A monopotent cell is one that can differentiate along only the endothelial cell lineage. A multipotent progenitor cell is one that can differentiate along either an endothelial or non-endothelial cell lineage. Without limiting the present invention in any way, it is generally thought that endothelial progenitor cells are monopotent. However, it would be appreciated by the person of skill in the art that under appropriate artificial conditions, particularly in vitro, a progenitor cell can sometimes be forced to differentiate along a lineage that would not occur naturally in vivo. It should therefore be understood that even if the isolated endothelial progenitor cells of the present invention are found to be capable of directed differentiation along non-endothelial cell lineages, provided that they have been isolated in accordance with the present invention and exhibit endothelial potential, they fall within the scope of the “endothelial progenitor cells” herein defined.

It should be understood that the endothelial progenitor cell populations and mesenchymal stem cell populations of the present invention may exhibit some variation in differentiative status within a single phenotypic profile. That is, within a single phenotypic profile, although the cells comprising that profile may substantially exhibit similar phenotypic and/or functional characteristics, there may nevertheless exhibit some differences. This may be apparent, for example, in terms of differences in the transcriptome profile or cell surface marker expression (other than the markers defined herein) of the cells that comprise the phenotypic profile in issue. For example, the CD45.sup.−/CD34.sup.+/CD31.sup.lo/−, the CD45.sup.−/CD34.sup.+/CD31.sup.− or the CD45.sup.−/CD34.sup.− cells may not represent a highly specific and discrete stage, but may be characterized by a number of discrete cellular subpopulations which reflect a transition or phase if one were to compare cells which have differentiated into this stage versus cells which are on the cusp of maturing out of this stage. This is typically characteristic, for example, by the onset of a sequential series of changes to gene expression, two or more of which are required to occur before the characteristic phenotypic profile defined herein is changed. Accordingly, the existence of cellular subpopulations within a single phenotypic profile of the present invention is encompassed.

According to this embodiment there is provided a method of isolating monopotent or multipotent mammalian endothelial progenitor cells said method comprising the steps of [0156] (i) isolating a mammalian cellular population; [0157] (ii) enriching for a subpopulation of the cells of step (i), which subpopulation expresses a CD45.sup.− phenotypic profile; [0158] (iii) enriching for a subpopulation of the CD45.sup.− cells derived from step (ii) which express a CD34.sup.+ phenotypic profile; and [0159] (iv) isolating the subpopulation of CD34.sup.+ cells derived from step (iii) which express a CD31.sup.lo/− phenotypic profile, to thereby isolate the endothelial progenitor cells.

In another embodiment, said endothelial progenitor cell is capable of differentiating to a vascular endothelial cell.

The subject endothelial progenitor cell and mesenchymal stem cells may be derived from any suitable mammalian tissue source including embryonic, cord, fetal, placental or post-natal tissue, such as adult tissue. To this end, reference to “mammal” includes humans, primates, livestock animals (e.g. horses, cattle, sheep, pigs, donkeys), laboratory test animals (e.g. mice, rats, guinea pigs), companion animals (e.g. dogs, cats) and captive wild animal (e.g. kangaroos, deer, foxes). Preferably, said mammal is a human.

Reference to a “mammalian cellular population” should be understood as a reference to a population of cells derived from a mammalian tissue source as defined above. The isolated cellular population (or “tissue sample” or “biological sample”--these terms being used interchangeably) should be understood as a reference to any sample of biological material which comprises cells and is derived from a mammal such as, but not limited to, cellular material (e.g. bone marrow or adipose aspirates), biological fluids (e.g. blood), tissue biopsy specimens (e.g. uterine biopsies), surgical specimens (e.g. hysterectomy tissue) or placental tissue.

The biological sample that is tested according to the method of the present invention may be tested directly or may require some form of treatment prior to testing. For example, a biopsy or surgical sample may require homogenization or other form of cellular dispersion prior to testing. Further, to the extent that the biological sample is not in liquid form, it may require the addition of a reagent, such as a buffer, to mobilize the sample and create a cell suspension. Alternatively, it may require some other form of pretreatment such a heparinization, where the sample is a whole blood sample, in order to prevent clotting.

The subject tissue (which includes reference to “cells”) may be a single cell suspension or a cell aggregate which has been freshly isolated from an individual (such as an individual who may be the subject of treatment) or it may have been sourced from a non-fresh source, such as from a culture (for example, where cell numbers were expanded and/or the cells were cultured so as to render them receptive to differentiative signals) or a frozen stock of cells which had been isolated at some earlier time point either from an individual or from another source. It should also be understood that the subject cells, prior to undergoing analysis in accordance with the present method, may have undergone some other form of treatment or manipulation, such as but not limited to enrichment or purification, modification of cell cycle status or the formation of a cell line. Accordingly, the subject cell may be a primary cell or a secondary cell. A primary cell is one that has been isolated from an individual. A secondary cell is one which, following its isolation, has undergone some form of in vitro manipulation prior to the application of the method of the invention.

In one particular embodiment, said mammalian cellular population is derived from umbilical cord blood or placenta, in particular post-parturition placenta.

According to this embodiment there is provided a method of isolating mammalian endothelial progenitor cells said method comprising the steps of: [0167] (i) isolating a mammalian placenta-derived cellular population; [0168] (ii) enriching for a subpopulation of the cells of step (i), which subpopulation expresses a CD45.sup.− phenotypic profile; [0169] (iii) enriching for a subpopulation of the CD45.sup.− cells derived from step (ii) which express a CD34.sup.+ phenotypic profile; and [0170] (iv) isolating the subpopulation of CD34.sup.+ cells derived from step (iii) which express a CD31.sup.lo/− phenotypic profile, to thereby isolate the endothelial progenitor cells.

In a related embodiment, the present invention is directed to a method of isolating mammalian mesenchymal stem cells said method comprising the steps of: [0172] (i) isolating a mammalian placenta-derived cellular population; [0173] (ii) enriching for a subpopulation of the cells of step (i), which subpopulation expresses a CD45.sup.− phenotypic profile; and [0174] (a) enriching for a subpopulation of the said CD45.sup.− cells derived from step (ii) which express a CD34.sup.+ phenotypic profile and isolating the subpopulation of said CD34.sup.+ cells which express a CD31.sup.− phenotypic profile and/or [0175] (b) isolating the subpopulation of CD45.sup.− cells derived from step (ii) which express a CD34.sup.− phenotypic profile, to thereby isolate the mesenchymal stem cells. In another embodiment, step (ii)-(iv) are performed sequentially.

Reference to “placenta-derived” cellular material should be understood as a reference to some or all of the heterogeneous population of cells that make up the placenta. Without limiting the present invention to any one theory or mode of action, in humans the placenta averages 22 cm in length and 2-2.5 cm in thickness, with the center being the thickest and the edges being the thinnest. It typically weighs approximately 500 grams. It exhibits a dark reddish-blue or crimson color and connects to the fetus by an umbilical cord of approximately 55-60 cm in length. The umbilical cord contains two umbilical arteries and one umbilical vein. The umbilical cord inserts into the chorionic plate. Vessels branch out over the surface of the placenta and further divide to form a network covered by a thin layer of cells. This results in the formation of villous tree structures. On the maternal side, these villous tree structures are grouped into lobules called cotyledons. In humans, the placenta usually has a disc shape, but size varies vastly between different mammalian species.

The placenta begins to develop upon implantation of the blastocyst into the maternal endometrium. The outer layer of the blastocyst becomes the trophoblast, which forms the outer layer of the placenta. This outer layer is divided into two further layers: the underlying cytotrophoblast layer and the overlying syncytiotrophoblast layer. The syncytiotrophoblast is a multinucleated continuous cell layer that covers the surface of the placenta. It forms as a result of differentiation and fusion of the underlying cytotrophoblast cells, a process that continues throughout placental development. The syncytiotrophoblast (otherwise known as syncytium) thereby contributes to the barrier function of the placenta. The placenta grows throughout pregnancy. Development of the maternal blood supply to the placenta is complete by the end of the first trimester of pregnancy (approximately 12-13 weeks).

In one embodiment, said placenta-derived cellular population is the cellular population of the cotyledons. Without limiting the present invention in any way, in one embodiment a post-parturition placenta is used, such as an intact placenta obtained following a caesarean section. The decidual component is dissected away in order to isolate the placental cotyledons. These cotyledons are then digested in a cocktail of enzymes, such as collagenase, dispase and DNAse, and thereafter filtered in order to obtain the starting population of cells for treatment in accordance with the method of the present invention.

It should be understood that in accordance with this particular embodiment of the present invention, one may use placenta at any stage of development. Although post-parturition placenta is most conveniently obtained, placentas from earlier stages of pregnancy may also be used, such as where a miscarriage or other termination of pregnancy occurs. As is discussed in more detail hereafter, placenta in particular and umbilical cord blood provide a good source of endothelial progenitor cells and mesenchymal stem cells which can now be efficiently isolated in accordance with the method developed by the present inventors. Accordingly, this provides the possibility of women routinely isolating and storing either placental/umbilical cord tissue or blood (for example) for future endothelial progenitor cell harvesting or else freshly harvesting and then freezing endothelial progenitor cells for future use. This therefore provides the possibility of either autologous endothelial progenitor cell treatment or, for individuals related to the donor, more closely MHC-matched endothelial progenitor cells than might otherwise be accessible. In both of these cases the donor endothelial progenitor cells are defined as being histocompatible with respect to the recipient of those cells.

In one embodiment there is therefore provided a method of isolating mammalian endothelial progenitor cells said method comprising the steps of: [0182] (i) isolating mammalian placenta cotyledon cellular material; [0183] (ii) enriching for a subpopulation of the cells of step (i), which subpopulation expresses a CD45.sup.− phenotypic profile; [0184] (iii) enriching for a subpopulation of the CD45.sup.− cells derived from step (ii) which express a CD34.sup.+ phenotypic profile; and [0185] (iv) isolating the subpopulation of CD34.sup.+ cells derived from step (iii) which express a CD31.sup.lo/− phenotypic profile, to thereby isolate the endothelial progenitor cells.

In still another embodiment there is provided a method of isolating mammalian mesenchymal stem cells said method comprising the steps of: [0187] (i) isolating mammalian placenta cotyledon cellular material; [0188] (ii) enriching for a subpopulation of the cells of step (i), which subpopulation expresses a CD45.sup.− phenotypic profile; and [0189] (a) enriching for a subpopulation of the CD45.sup.− cells derived from step (ii) which express a CD34.sup.+ phenotypic profile and isolating the subpopulation of said CD34.sup.+ cells) which express a CD31.sup.− phenotypic profile; and/or [0190] (b) isolating the subpopulation of CD45.sup.− cells derived from step (ii) which express a CD34.sup.− phenotypic profile, to thereby isolate the mesenchymal stem cells.

It would be appreciated that the phenotypic characterization of the cellular population of the present invention is a significant development since the identification of a reliable marker profile in the context of the endothelial cell hierarchy has been elusive. Without limiting the present invention to any one theory or mode of action: (i) CD45 is a protein tyrosine phosphatase, receptor type, C also known as PTPRC, which is an enzyme that, in humans, is encoded by the PTPRC gene. CD45 was originally called leukocyte common antigen or T200. The protein encoded by this gene is a member of the protein tyrosine phosphatase (PTP) family. PTP are known to be signaling molecules that regulate a variety of cellular processes including cell growth, differentiation, mitotic cycle, and oncogenic transformation. The CD45 family consists of multiple members that are all products of a single complex gene. This gene contains 34 exons and three exons of the primary transcripts are alternatively spliced to generate up to eight different mature mRNAs and after translation eight different protein products. These three exons generate the RA, RB and RC isoforms. Various isoforms of CD45 exist: CD45RA, CD45RB, CD45RC, CD45RAB, CD45RAC, CD45RBC, CD45RO, CD45R (ABC). [0194] (ii) CD34 is a cell surface glycoprotein and functions as a cell-cell adhesion factor. It may also mediate the attachment of stem cells to bone marrow extracellular matrix or directly to stromal cells. CD34 is also the name for the human gene that encodes the protein. The CD34 protein is a member of a family of single-pass transmembrane sialomucin proteins that show expression on early hematopoietic and vascular-associated tissue. Cells expressing CD34 are normally found in the umbilical cord and bone marrow as hematopoietic cells, a subset of mesenchymal stem cells, endothelial progenitor cells, endothelial cells of blood vessels but not lymphatics (except pleural lymphatics), mast cells, a sub-population dendritic cells (which are factor XIIIa negative) in the interstitium and around the adnexa of dermis of skin, as well as cells in soft tissue tumors like DFSP, GIST, SFT, HPC. (iii) Platelet endothelial cell adhesion molecule (PECAM-1) also known as CD31 is a protein that in humans is encoded by the PECAM1 gene found on chromosome 17. PECAM-1 is found on the surface of platelets, monocytes, neutrophils, and some types of T−cells, and makes up a large portion of endothelial cell intercellular junctions. The encoded protein is a member of the immunoglobulin superfamily and is likely involved in leukocyte migration, angiogenesis, and integrin activation. CD31 is normally found on endothelial cells, platelets, macrophages and Kupffer cells, granulocytes, T/NK cells, lymphocytes, megakaryocytes, osteoclasts and neutrophils.

In the context of the present invention, it should be understood that reference to “CD45”, “CD34” and “CD31” is a reference to all forms of these molecules and to functional fragments, mutants or variants thereof. It should also be understood to include reference to any isoform that may arise from alternative splicing of CD45, CD34 and CD31 mRNA or isomeric or polymorphic forms of these molecules. Reference to “phenotypic profile” should be understood as a reference to the presence or absence of the transcription of the genes encoding the subject markers and/or the cell surface expression of the expression product translated therefrom. It should be appreciated that although most cells falling within the scope of the claimed endothelial progenitor cell populations will be characterized by the presence or absence of the subject marker as a cell surface anchored expression product, some cells falling within the defined populations may initially exhibit changes only at the transcriptome level, such as when the transcription of a given marker has been upregulated but may not yet have resulted in a cell surface anchored expression product. In general, cells which progress to a new differentiative stage will transiently exhibit gene expression changes which are not yet evident in the context of changes to levels of an expression product. However, these cells nevertheless fall within the scope of the claimed cellular populations, although they will not be isolatable by the method defined herein until such time as cell surface marker expression occurs.

It should also be appreciated that although the endothelial progenitor and mesenchymal stem cell populations of the present invention are characterized by the defined phenotypic profiles, these cells will express a range of other intracellular and/or cell surface markers which are not relevant in terms of phenotypically characterizing and isolating the cellular population of interest. Still further, to the extent that a given endothelial progenitor cell population of the present invention comprises a range of subpopulations, these subpopulations may exhibit variations in the expression of intracellular or cell surface markers other than those of the profiles defined herein. Although the CD45 and CD34 cell surface markers are defined by reference to the presence or absence of the marker on the cell surface, the expression of CD31 is defined by reference to the level of expression, specifically a low level of expression (herein referred to as “CD31.sup.lo/−”). In the embodiment of the invention exemplified herein, the “CD31.sup.lo/−” subpopulation is based on defining a FACS gate based on an isotype control. In this exemplified embodiment, only the isotype control for CD31 is used and all other antibodies are kept equal. Three populations are seen based on CD31 level of expression. The first is negative for CD31 that gives rise to the fetal mesenchymal stem cells. The second population that gives rise to the endothelial progenitor cells is where the positive gate starts. Finally there is a CD31.sup.+ population that has limited proliferative capacity. It would be appreciated by the skilled person that the specific manner in which the analysis is set up and the logs that are used can vary according to the voltage of the FACS. However, these parameters can be established as a matter of routine procedure by the skilled person. The term “lo/−” as used in relation to CD31.sup.lo/− is well known in the art and refers to the expression level of CD31, in that the expression level of this cell surface marker is low by comparison with the expression level of that marker in the population of cells being analyzed as a whole. The term “lo” in relation to CD31.sup.lo refers to a distinct cell or population of cells that expresses CD31 at a lower level than one or more other distinct cells or populations of cells. Thus, the terms CD31.sup.lo/− and CD31.sup.lo are used interchangeably herein to refer to the endothelial progenitor cells resulting from the subject isolation methods. In specific embodiments, the level of CD31 expressed by a CD31.sup.lo cell or population of cells is less than 50% (and less than 49% to no less than 1% and all integer percentages in between, suitably less than 40% to no less than 1% and all integer percentages in between, suitably less than 30% to no less than 1% and all integer percentages in between, suitably less than 20% to no less than 1% and all integer percentages in between, even more suitably less than 10% to no less than 1% and all integer percentages in between) of the level of CD31 expressed by a HUVEC or HUVEC population. The terms “+” and “−” are well known in the art and refer to the expression level of the cell marker of interest, in that the expression level of the cell marker corresponding to “+” is high or intermediate and the expression level of the cell marker corresponding to “−” is null. Cells in the top 2, 3, 4, or 5% of staining intensity are often designated “hi”, with those falling in the top half of the population categorized as being “+”. Those cells falling below 50% of fluorescence intensity are designated as “lo” cells and below 1% as “−” cells.

The term “high” or “hi” or “bright” is well known in the art and refers to the expression level of the cell marker of interest, in that the expression level of the cell marker is high by comparison with the expression level of that cell marker in the population of cells being analyzed as a whole:

In terms of the CD45.sup.−CD34.sup.− mesenchymal stem cell population, this population gives rise to a significant population of maternal mesenchymal stem cells. Without limiting the present invention to any one theory or mode of action, it is thought that this cellular fraction is in fact heterogeneous. However, upon in vitro culture of these cells under conditions appropriate for mesenchymal stem cells, cells other than mesenchymal cells do not grow, thereby leading to the generation of a population of predominantly mesenchymal stem cells which are of maternal origin.

The present invention also encompasses an isolated population of cells containing endothelial progenitor cells and/or mesenchymal stem cells as broadly described above and elsewhere herein. As used herein, “and/or” refers to and encompasses any and all possible combinations of one or more of the associated listed items, as well as the lack of combinations when interpreted in the alternative (or). In specific embodiments, the isolated cell population is a population in which the EPC and/or MSC are enriched over a starting cell population, i.e., that the EPC and/or MSC are present in number or as a percentage of the cells that is greater than a starting cell population or sample. Cell populations enriched for EPC are suitably produced using the methods for producing EPC and/or MSC as described herein.

In certain embodiments, the EPC in the isolated population are 10% or more of the cells in the population, including 20% or more, 30% or more, 40% or more, 50% or more, 70% or more, 80% or more, 90% or more, 97% or more, up to and include 100% of the cells in the isolated population.

In certain embodiments, the MSC in the isolated population are 10% or more of the cells in the population, including 20% or more, 30% or more, 40% or more, 50% or more, 70% or more, 80% or more, 90% or more, 97% or more, up to and include 100% of the cells in the isolated population.

The EPC of the subject invention, which are suitably derived from placenta, can be characterized by their gene expression pattern relative to the pattern of gene expression in EPC derived from umbilical cord blood (UCB). The terms “expression” or “gene expression” refer to production of RNA only or production of RNA and translation of RNA into proteins or polypeptides. Detection of either types of gene expression is encompassed by the present invention.

In particular, the EPC of the present invention differentially express the genes set out in Table 1 relative to the expression of the corresponding genes in UBC-derived EPC.

Any medium capable of supporting MSC in vitro may be used to culture the MSC. Media formulations that can support the growth of MSC include, but are not limited to, Dulbecco's Modified Eagle's Medium (DMEM), alpha modified Minimal Essential Medium (.alpha.MEM), and Roswell Park Memorial Institute Media 1640 (RPMI Media 1640) and the like. Said media and conditions for culture of MSC—and by virtue of the invention MSC are known in the art. Typically, up to 20% fetal bovine serum (FBS) or 1-20% horse serum is added to the above medium in order to support the growth of MSC. A defined medium, however, also can be used if the growth factors, cytokines, and hormones necessary for culturing MSC are provided at appropriate concentrations in the medium. Media useful in the methods of the invention may contain one or more compounds of interest, including, but not limited to, antibiotics, mitogenic or differentiation compounds useful for the culturing of MSC. The cells may be grown at temperatures between 27.degree. C. to 40.degree. C., preferably 31.degree. C. to 37.degree. C., and more preferably in a humidified incubator. The carbon dioxide content may be maintained between 2% to 10% and the oxygen content may be maintained between 1% and 22%. The invention, however, should in no way be construed to be limited to any one method of isolating and culturing MSC. Rather, any method of isolating and culturing MSC should be construed to be included in the present invention.

Antibiotics which can be added into the medium include, but are not limited to, penicillin and streptomycin. The concentration of penicillin in the culture medium, in a non-limiting embodiment, is about 10 to about 200 units per ml. The concentration of streptomycin in the culture medium is, in a non-limiting embodiment, about 10 to about 200.mu.g/ml.

MSC which express increased amounts of at least one anti-apoptotic protein may be administered to an animal in an amount effective to provide a therapeutic effect. The animal may be a mammal, including but not limited to, human and non-human primates.

The MSC can be suspended in an appropriate diluent. Suitable excipients for injection solutions are those that are biologically and physiologically compatible with the MSC and with the recipient, such as buffered saline solution or other suitable excipients. The composition for administration can be formulated, produced, and stored according to standard methods complying with proper sterility and stability. The MSC may have one or more genes modified or be treated such that the modification has the ability to cause the MSC to self-destruct or “commit suicide” because of such modification, or upon presentation of a second drug (eg., a prodrug) or signaling compound to initiate such destruction of the MSC.

In one embodiment of the invention, conditioned media from mesenchymal stem cells is utilized to treat adverse effects of cancer chemotherapy and radiation therapy, including mucositis. In one specific embodiment, exosomes derived from mesenchymal stem cells are utilized in a mouthwash to treat mucositis. In another embodiment, exosomes or other components of mesenchymal stem cell supernatant, or mesenchymal stem cell lysates are utilized to treat mucositis or hair loss by topical administration. In another embodiment, mesenchymal stem cells, or conditioned media thereof, or lysates thereof are utilized to treat cancer cachexia.

In one embodiment of the invention, use of mesenchymal stem cells, and/or exosomes derived thereof, is disclosed as a means of treating post traumatic stress disorder (PTSD). A description of PTSD is provided to enable one of skill in the art to practice the invention. PTSD is a devastating psychiatric condition associated with tremendous emotional and financial costs to the health care system[86]. It is estimated that approximately 6.8% of Americans will suffer from PTSD at one point in their lives [87], with significantly higher proportions in war veterans [88-90]. Manifestations of PTSD include three major mental alterations, formally classified as four clusters of psychiatric symptoms. The first, termed “re-experiencing”, involves the emotional and perceptual reliving of traumatic event either spontaneously or in response to triggers that remind one of the event because they bear some similarity to the original circumstance. The second symptom cluster, termed “avoidance and numbing”, involves the tendency to social isolation and reduced ability to experience positive emotions in relationships with others. The third cluster involves hypervigilance about one's surroundings, sleep disturbance, anxiety, and lack of ability to maintain anger control, often leading physical violence. In the DSM-V, a 4th cluster of symptoms, termed ‘negative alterations in cognitions and mood’, has been added. It incorporates several symptoms previously included in the DSM-IV avoidance and numbing cluster, and adds persistent distorted blame of self or others, and persistent negative emotional state as new symptoms, based on empirical data on the phenomenology of the condition published since DSM-IV [91].

In a US government study it was estimated that PTSD in veterans of the Iraq and Afghanistan wars cost the American Health Care System 2.8 billion dollars annually [92, 93]. Severely depressed quality of life is reported in PTSD patients [94-96], including clinical depression [97-99], deterioration of marital and family relationships [95, 100], inability to maintain employment [94, 101], exaggerated proclivity towards substance abuse [102], general medical illnesses such as increased risk of heart failure [103], suicidal tendencies and execution [104], and early death [105].

Currently the main treatment interventions for PTSD include psychotropic medications and/or psychotherapy. Antidepressants are commonly prescribed [106, 107]. The selective serotonin reuptake inhibitor (SSRI) antidepressants, sertraline and paroxetine, are the only US Food and Drug Administration (FDA) approved medications for the condition. Although positive effects were reported in the pivotal study, it is important to mention that effects where overall not staggering and that less than 10% of the patients in the trials leading to FDA approval were combat PTSD sufferers [108-111]. In fact, two clinical studies evaluating SSRIs in combat related PTSD demonstrated no significant benefit [112, 113], this in part, is associated with recent recommendations against using SSRIs in treatment of PTSD [114]. The selective serotonin and norepinephrine reuptake inhibitor Venlafaxine, and the sympatholytic alpha blocker Prazosin have demonstrated some efficacy in open label trials [115-117]. Unfortunately these agents have not been demonstrated efficacious in large randomized controlled trials. Some commonly used pharmacologic strategies, including second-generation antipsychotic augmentation of unsuccessful antidepressant therapy, as well as divalproex and bupropion, have failed to separate from placebo in RCTs with combat vets, and one very commonly used medication class—benzodiazepines—while widely used in clinical settings, has no supporting evidence, and is described as not effective and potentially harmful in the recent National Center for PTSD (NCPTSD) treatment guideline [118]. According to the same guidelines the use of Exposure therapy is recommended. One promising treatment approach, exposure therapy, follows from the hypothesis that PTSD is a disorder of emotional learning [119]. Specifically, in exposure therapy the goal is to relive a traumatic event within a safe context in order to alter the emotional manifestations associated with the event. Since PTSD is the only psychiatric disorder that requires the occurrence of an external event as a prerequisite to diagnosis,this event provides the context for learning. It is known that across species, pairing a neutral stimulus with an aversive one leads to the learning of a conditioned fear response. In humans with PTSD, the matrix of sensory stimuli embedded in the traumatic memory serve as cues that evoke a conditioned fear response in the absence of the original trauma (the unconditioned aversive stimuli). This conditioned fear response manifests as avoidance of trauma-associated cues,including thoughts, feelings, or sensory (eg, olfactory) reminders and the experience of emotional distress when faced with these reminders. A conditioned fear response can be initially adaptive, but it should extinguish when the conditioned cues are no longer accompanied by actual risk of danger. Individuals with PTSD have not learned that the stimuli associated with their trauma are now safe. Thus, PTSD may manifest with a persisting conditioned fear response independent of the original trauma and difficulty learning that stimuli previously associated with a trauma are no longer present [120]. Through exposure therapy, the psychiatrist attempts to correct the negative associations in PTSD and accelerate extinction of emotional memory charged with negative consequences associated with PTSD.

Animal and human studies robustly demonstrate that fear is extinguished experimentally by repeatedly presenting the conditioned stimulus in the absence of the aversive stimulus, a process that has been associated with amygdala depotentiation [121, 122]. In humans, this model translates into repeatedly re-experiencing the traumatic memory in a safe environment (absence of the aversive stimuli) until the fear is extinguished. This process is hypothesized to be the mechanism of action in exposure therapy, the treatment with the strongest empirical evidence for PTSD [123].

One of the important aspects of exposure therapy is the mechanism by which during the retrieval of the memory, the memory becomes sensitive to manipulation, before reconsolidation. If manipulation is induced during the reconsolidation phase, the memory may be lost, or its emotional significance may be altered. The experimental manipulation of memory reconsolidation was resurrected after a 30-year hiatus [124], by studies from Nader et al. who described the disruption of Pavlovian fear memories by anisomycin administered after retrieval. The principle of reconsolidation manipulation is based on the findings that ‘New’ memories are initially labile and sensitive to disruption before being consolidated into stable long-term memories. The process of memory reconsolidation appears to involve new protein synthesis, particularly in the areas of the brain known as the lateral and basal nuclei of the amygdala (LB A) that are believed to be a site of memory storage in fear learning. This has been previously demonstrated by experiments in which injections of the protein synthesis inhibitor anisomycin into the LBA shortly after training prevents consolidation of fear memories [125, 126]. The experiments by Nader et al. showed that consolidated fear memories, when reactivated during retrieval, return to a labile state in which infusion of anisomycin shortly after memory reactivation produces amnesia on later tests, regardless of whether reactivation was performed 1 or 14 days after conditioning. Treatment with anisomycin, in the absence of memory reactivation, left memory intact. Consistent with a time-limited role for protein synthesis production in consolidation, delay of the infusion until six hours after memory reactivation produced no amnesia. These data showed that consolidated fear memories, when reactivated, return to a labile state that requires de novo protein synthesis for reconsolidation [127]. This study demonstrated first, that consolidated memories could be “erased” after retrieval, and second, that mechanistically, this so-called “reconsolidation” process resembled the original consolidation in its requirement for protein synthesis.

Although the use of protein synthesis inhibitors is not clinically useful, various pharmacotherapeutics are being developed for augmentation of the extinction learning process that may occur during exposure therapy. D-cycloserine (DCS) (Seromycin) is a partial agonist at the N-methyl-D-aspartate (NMDA) receptor, a member of the glutamate receptor family, which has an essential role in mediating learning and memory. Both fear learning and extinction are blocked by antagonists at the glutamatergic NMDA receptor.

The importance of the NMDA system in extinction is suggested by numerous studies [128-131]. In one experimental system, Zimmerman and Maren assessed the role of NMDA receptors in the central nucleus of the amygdala (CEA), which is known to be involved in the acquisition of conditional fear, but it is not known whether they play a role in fear extinction. Infusion of glutamate receptor antagonists into the basolateral complex of the amygdala (BLA) or CEA prior to the extinction of fear to an auditory conditioned stimulus (CS) in rats was performed. Infusion of the alpha-amino-3-hydroxyl-5-methyl-4-isoxazole-propionate (AMPA) receptor antagonist, 2,3-dihydroxy-6-nitro-7-sulfamoyl-benzo[f]quinoxaline-2,3-dione (NBQX), into either the CEA or BLA impaired the expression of conditioned freezing to the auditory CS, but did not impair the formation of a long-term extinction memory to that CS. In contrast, infusion of the N-methyl-D-aspartate (NMDA) receptor antagonist, D,L-2-amino-5-phosphonopentanoic acid (APV), into the amygdala, spared the expression of fear to the CS during extinction training, but impaired the acquisition of a long-term extinction memory. Importantly, only APV infusions into the BLA impaired extinction memory. These results reveal that AMPA and NMDA receptors within the amygdala make dissociable contributions to the expression and extinction of conditioned fear, respectively [132].

Manipulation of extinction has been successfully performed in rodent models. For example, Ledgerwood et al. established a system where rats received 5 light-shock pairings as conditioning. The following day, rats received 6 light-alone presentations in order to induce extinction. Twenty-four hours later, rats received 1 light-alone presentation (test). Subcutaneous DCS injection before or after extinction training significantly enhanced extinction, and the dose-response curve for this effect was linear. Increasing the delay of DCS administration after extinction training led to a linear decrease in the facilitatory effect. The effect of systemic administration was replicated by intra-basolateral amygdala infusion. These results suggested that DCS facilitates extinction of conditioned freezing by acting on consolidation processes partly mediated by the basolateral amygdala [133].

In one embodiment of the invention, mesenchymal stem cells, or exosomes derived thereof, are administered before, during, or after exposure therapy to provide inhibition of NMDA receptor activity. Without being bound to theory, the invention teaches that mesenchymal stem cells, or exosomes derived thereof, may be administered at a sufficient concentration and frequency to augment the therapeutic effects of exposure therapy, in part, through inhibition of NMDA receptor signaling. In another embodiment of the invention, mesenchymal stem cells are administered without exposure therapy.

Various aspects of the invention of the invention relating to the above are enumerated in the following paragraphs:

Aspect 1. A method of protecting non-neoplastic cells from cellular damaging effects of a brain cancer directed therapy, said method comprising the steps of: a) obtaining a cell with regenerative potential; and b) administering said cell in a manner to allow chemotaxis and/or proximity to non-malignant brain tissue at a concentration and frequency sufficient to provide selective protection of non-malignant tissue from effects of chemotherapy and/or radiation therapy.

Aspect 2. The method of aspect 1, wherein said brain cancer directed therapy comprises therapies selected from a group comprising of: a) radiation therapy; b) chemotherapy; c) surgery; d) metabolic therapy; and e) immunotherapy.

Aspect 3. The method of aspect 1, wherein said regenerative cell is selected from a group comprising of; a) T cells; b) B cells; c) progenitor cells; and d) stem cells.

Aspect 4. The method of aspect 3, wherein said progenitor cells are selected from a group comprising of; a) myeloid progenitors; b) lymphoid progenitors; c) mesenchymal progenitors; d) fetal liver progenitors.

Aspect 5. The method of aspect 4, wherein said progenitor cells are mesenchymal stem cells.

Aspect 6. The method of aspect 5, wherein said mesenchymal stem cells express proteins selected from a group comprising of ; a) CD73; b) CD90; and c) CD105

Aspect 7. The method of aspect 1, wherein said regenerative cells selectively home to brain tissue that has been irradiated.

Aspect 8. The method of aspect 1, wherein said regenerative cells selectively home to brain tissue that has been treated with chemotherapy.

Aspect 9. The method of aspect 1, wherein said regenerative cells selectively home to brain tissue that has been treated with immunotherapy.

Aspect 10. The method of aspect 1, wherein said regenerative cells selectively home to brain tissue that has been treated with metabolic therapy.

Aspect 11. The method of aspect 5, wherein said mesenchymal stem cells possess ability to secrete cytokines selected from a group comprising of; a) IFN-gamma; b) TNF-alpha; c) IL-2; d) IL-7; e) IL-12; f) IL-15; g) IL-17; h) IL-18; i) IL-21; j) IL-23; k) IL-27; l) IL-33; m) HMGB-1; and n) TRAIL.

Aspect 12. The method of aspect 11, wherein said mesenchymal stem cells are cultured under conditions allowing for enhanced production of cytokines selected from a group comprising of; a) IFN-gamma; b) TNF-alpha; c) IL-2; d) IL-7; e) IL-12; f) IL-15; g) IL-17; h) IL-18; i) IL-21; j) IL-23; k) IL-27; l) IL-33; m) HMGB-1; and n) TRAIL.

Aspect 13. The method of aspect 11, wherein said mesenchymal stem cells are transfected with genes allowing for enhanced production of cytokines selected from a group comprising of; a) IFN-gamma; b) TNF-alpha; c) IL-2; d) IL-7; e) IL-12; f) IL-15; g) IL-17; h) IL-18; i) IL-21; j) IL-23; k) IL-27; l) IL-33; m) HMGB-1; and n) TRAIL.

Aspect 14. The method of aspect 13, wherein said transfection of said genes is performed in a manner whereas induction of gene expression occurs in an inducible manner.

Aspect 15. The method of aspect 14, wherein said gene expression in an inducible manner is provided by a vector comprising a polynucleotide encoding a gene switch, said gene switch comprising (1) at least one transcription factor sequence, wherein said at least one transcription factor sequence encodes a ligand-dependent transcription factor comprising an ecdysone receptor ligand binding domain, operably linked to a promoter, and (2) a polynucleotide encoding a polypeptide at least 85% identical to the wild type human therapeutic polypeptide sequence linked to a promoter which is activated by said ligand-dependent transcription factor wherein following administration of said in vitro engineered ERC to a mammal with a disease, and a first administration of a ligand to said mammal less than 48 hours after said in vitro ERC are administered, wherein said ligand is thereafter administered daily for a period of 2 to 30 days, the therapeutic effect of said engineered in said mammal is reduced.

Aspect 16. The method of aspect 15, wherein said vector is vector selected from a group of vectors comprising of: a) a lentiviral vector; b) adenoviral vector; c) an adeno-associated viral vector.

Aspect 17. The method of aspect 15, wherein said polynucleotide encoding a gene switch comprises a first transcription factor sequence and a second transcription factor sequence under the control of a promoter, wherein the proteins encoded by said first transcription factor sequence and said second transcription factor sequence interact to form a protein complex which functions as a ligand-dependent transcription factor.

Aspect 18. The method of aspect 17, wherein said first transcription factor and said second transcription factor are connected by an internal ribosomal entry site.

Aspect 19. The method of aspect 15, wherein said polynucleotide encoding a gene switch comprises a first transcription factor sequence under the control of a first promoter and a second transcription factor sequence under the control of a second promoter, wherein the proteins encoded by said first transcription factor sequence and said second transcription factor sequence interact to form a protein complex which functions as a ligand-dependent transcription factor.

Aspect 20. The method aspect 15, wherein said ligand is selected from the group consisting of RG-115819, RG-115932, and RG-115830.

Aspect 21. The method of aspect 15, wherein said ligand is an amidoketone or oxadiazoline.

Aspect 22. The method of aspect 15, wherein said polynucleotide sequence encoding a gene switch comprises a polynucleotide sequence encoding a VP-16 transactivation domain.

Aspect 23. The method of aspect 22, wherein said polynucleotide sequence encoding a gene switch comprises a polynucleotide sequence encoding a GAL-4 DNA binding domain.

Aspect 24. The method of aspect 5, wherein said mesenchymal stem cells are engineered for enhanced vivo persistence through transfection of a therapeutic peptide sequence encoding an anti-apoptotic gene.

Aspect 25. The method of aspect 24, wherein said anti-apoptotic gene is selected from a group comprising of: obestatin, XIAP, survivin, BCL-2, BCL-XL, GATA-4, IGF-1, EGF, heme-oxygenase-1, NF-kB, akt, pi3-k, and epha-2.

Aspect 26. The method of aspect 25, wherein said mesenchymal stem cell persistence is augmented through transfection of a gene construct capable of inducing RNA interference directed against a molecule associated with induction of apoptosis.

Aspect 27. The method of aspect 26, wherein said molecules associated with induction of apoptosis are selected from a group comprising of: Fas, FasL, CASP1 (ICE), CASP10 (MCH4), CASP14, CASP2, CASP3, CASP4, CASP5, CASP6, CASP7, CASP8, CASP9, CFLAR (CASPER), CRADD, PYCARD (TMS1/ASC), ABL1, AKT1, BAD, BAK1, BAX, BCL2L11, BCLAF1, BID, BIK, BNIP3, BNIP3L, CASP1 (ICE), CASP10 (MCH4), CASP14, CASP2, CASP4, CASP6, CASP8, CD70 (TNFSF7), CIDEB, CRADD, FADD, FASLG (TNFSF6), HRK, LTA (TNFB), NOD1 (CARD4), PYCARD (TMS1/ASC), RIPK2, TNF, TNFRSF10A, TNFRSF10B (DRS), TNFRSF25 (DR3), TNFRSF9, TNFSF10 (TRAIL), TNFSF8, TP53, TP53BP2, TRADD, TRAF2, TRAF3, and TRAF4.

Aspect 28. The method of aspect 27, wherein said mesenchymal stem cells are endowed with ability to differentiate into cells of the neuronal lineage at a specified time point associated with homing to neuronal tissue.

Aspect 29. The method of aspect 5, wherein said mesenchymal stem cells are administered intravenously.

Aspect 30. The method of aspect 5, wherein said mesenchymal stem cells are administered intrathecally.

Aspect 31. The method of aspect 5, wherein said mesenchymal stem cells are administered intraventricularly.

Aspect 32. The method of aspect 5, wherein said mesenchymal stem cells are administered stereotactically.

Aspect 33. The method of aspect 28, wherein differentiation into the neural lineage is accomplished by transfection of a therapeutic polypeptide sequence selected from a group of polypeptide sequences comprising of: ADCYAP1R1, ARTN, BDNF, CD40 (TNFRSF5), CNTF, CNTFR, CRHBP, CRHR1, CRHR2, FRS2, FRS3, FUS, GDNF, GFRA1, GFRA2, GFRA3, GMFB, GMFG, MAGED1, MT3, NF1, NGF, NGFR, NGFRAP1, NR1I2, NRG1, NRG2, NTF3, NTF4, NTRK1, NTRK2, PSPN, PTGER2, TFG, TRO, VGF.

Aspect 34. The method of aspect 33, wherein said polypeptide sequences are utilized to induce differentiation of endogenous neural progenitors as a result of paracrine or systemic effects of said mesenchymal stem cell expressing said polypeptide sequences.

Aspect 35. The method of aspect 5, wherein said mesenchymal stem cells are modified to possess enhanced angiogenic activity, said angiogenic activity selectively associated with stimulation of non-malignant neural tissue regeneration, wherein said enhanced ability to stimulate angiogenesis is accomplished through transfection with an angiogenic polypeptide.

Aspect 36. The method of aspect 35, wherein said angiogenic polypeptide is selected from a group comprising of: activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, collagen receptors .alpha..sub. 1.beta..sub.1 and .alpha..sub. 2.beta..sub. 1, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin and fibronectin receptor .alpha.5.beta. 1, Factor X, HB-EGF, HBNF, HGF, HUAF, heart derived inhibitor of vascular cell proliferation, Ill, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-.beta., PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-.beta., and TGF-.beta. receptors, TIMPs, TNF-alphatransferrin, thrombospondin, urokinase, VEGF-A, VEGF-B, VEGF-C, VEGF-D, VEGF-E, VEGF, VEGF.sub.164, VEGI, EG-VEGF.

Aspect 37. The method of aspect 5, wherein said mesenchymal stem cell is endowed with enhanced ability to stimulate immunity.

Aspect 38. The method of aspect 37, wherein said enhanced ability to stimulate immunity is accomplished through transfection with an immunomodulatory polypeptide.

Aspect 39. The method of aspect 38, wherein said immunomodulatory polypeptide is selected from a group comprising of: ABCF1, BCL6, C3, C4A, CEBPB, CRP, ICEBERG, IL1R1, IL1RN, IL8RB, LTB4R, TOLLIP, IFNA2, ILlORA, ILlORB, IL13, IL13RA1, IL5RA, IL9, IL9R, CD40LG (TNFSF5), IFNA2, IL17C, IL1A, IL1B, IL1F10, IL1F5, IL1F6, IL1F7, IL1F8, IL1F9, IL22, IL5, IL-6, IL8, IL9, IL-18, IL-33, LTA, LTB, MIF, SCYE1, SPP1, TNF, CCL13 (mcp-4), CCR1, CCR2, CCR3, CCR4, CCR5, CCR6, CCR7, CCR8, CCR9, CX3CR1, IL8RA, XCR1 (CCXCR1), C5, CCL1 (I-309), CCL11 (eotaxin), HMGB1, IL-2. IL-12, IL-17, IL33. CCL13 (mcp-4), CCL15 (MIP-1d), CCL16 (HCC-4), CCL17 (TARC), CCL18 (PARC), CCL19, CCL2 (mcp-1), CCL20 (MIP-3a), complement components C3, and C5, 2,3 alpha gal, CCL21 (MIP-2), CCL23 (MPIF-1), CCL24 (MPIF-2/eotaxin-2), CCL25 (TECK) , CCL26, CCL3 (MIP-1a), CCL4 (MP-1b), CCLS (RANTES), CCL7 (mcp-3), CCL8 (mcp-2), CXCL1, CXCL10 (IP-10), CXCL11 (I-TAC/IP-9), CXCL12 (SDF1), CXCL13, CXCL14, CXCL2, CXCL3, CXCL5 (ENA-78/LIX), CXCL6 (GCP-2), CXCL9, IL13, and IL8.

Aspect 40. A method of protecting non-malignant neural tissue from effects of an anticancer therapeutic targeting the brain, said method comprising the steps of; a) obtaining a regenerative cell population; b) collecting exosomes from said regenerative cell population; c) administering said exosomes in a patient receiving an anticancer therapeutic targeting the brain.

Aspect 41. The method of aspect 40, wherein said regenerative cell population is selected from a group comprising of; a) T cells; b) B cells; c) progenitor cells; and d) stem cells.

Aspect 42. The method of aspect 41, wherein said progenitor cells are selected from a group comprising of; a) myeloid progenitors; b) lymphoid progenitors; c) mesenchymal progenitors; d) fetal liver progenitors.

Aspect 43. The method of aspect 42, wherein said progenitor cells are mesenchymal stem cells.

Aspect 44. The method of aspect 43, wherein said mesenchymal stem cells express proteins selected from a group comprising of ; a) CD73; b) CD90; and c) CD105

Aspect 45. The method of aspect 40, wherein said exosomes express phosphotidylserine.

Aspect 46. The method of aspect 40, wherein said exosomes are microvesicles.

Aspect 47. The method of aspect 1, wherein said cell with regenerative potential is derived from placental tissue.

Aspect 48. The method of aspect 47, wherein said cell with regenerative potential is an endothelial cell population.

Aspect 49. The method of aspect 48, wherein said endothelial cell population is an endothelial progenitor cell.

Aspect 50. The method of aspect 49, wherein said endothelial progenitor cell is derived by a method comprising the steps of: (i) isolating a mammalian cellular population; (ii) enriching for a subpopulation of the cells of step (i), which subpopulation expresses a CD45.sup.− phenotypic profile; (iii) enriching for a subpopulation of the CD45.sup.− cells derived from step (ii) which express a CD34.sup.+ phenotypic profile; and (iv) isolating the subpopulation of CD34.sup.+ cells derived from step (iii) which express a CD31.sup.lo/− phenotypic profile, to thereby isolate the endothelial progenitor cells.

Aspect 51. The method of aspect 47, wherein said regenerative cell derived from placental tissue is a mesenchymal stem cell.

Aspect 52. The method of aspect 51, wherein said mesenchymal stem cell is a mesenchymal progenitor cell.

Aspect 53. The method of aspect 51, wherein said mesenchymal progenitor cell expresses markers selected from a group comprising of; a) NANOG; b) OCT-4; c) SSEA-4; and d) stem cell factor receptor.

Aspect 54. The method of aspect 51, wherein said mesenchymal stem cell is isolated by a method comprising the steps of: (i) isolating a mammalian cellular population; (ii) enriching for a subpopulation of the cells of step (i), which subpopulation expresses a CD45.sup.− phenotypic profile; and (a) enriching for a subpopulation of the CD45.sup.− cells derived from step (ii) which express a CD34.sup.+ phenotypic profile and isolating the subpopulation of said CD34.sup.+ cells which express a CD31.sup.− phenotypic profile and/or (b) isolating the subpopulation of CD45.sup.− cells derived from step (ii) which express a CD34.sup.− phenotypic profile, to thereby isolate the mesenchymal stem cells.

Aspect 55. The method of aspect 51, wherein said mesenchymal stem cells are isolated by a method comprising the sequential steps of: (i) isolating a mammalian cellular population; (ii) enriching for a subpopulation of the cells of step (i), which subpopulation expresses a CD45.sup.− phenotypic profile; and (a) enriching for a subpopulation of the CD45.sup.− cells derived from step (ii) which express a CD34.sup.+ phenotypic profile and isolating the subpopulation of said CD34.sup.+ cells which express a CD31.sup.− phenotypic profile; and/or (b) isolating the subpopulation of CD45.sup.− cells derived from step (ii) which express a CD34.sup.− phenotypic profile, to thereby isolate the mesenchymal stem cells.

Aspect 56. The method according aspect 54, wherein the CD31.sup.− population is a fetal CD31.sup.− population and said mesenchymal stem cells are fetal mesenchymal stem cells.

Aspect 57. The method according to aspect 54, wherein the CD45.sup.−/CD34.sup.− mesenchymal stem cells are maternal stem cells.

Aspect 58. A method of therapeutically and/or prophylactically treating a neurological condition in a mammal, said method comprising administering to said mammal an effective number of endothelial progenitor cells or partially or fully differentiated EPC-derived cells, which endothelial progenitor cells have been isolated according to the method of aspect 53.

Aspect 59. A method of facilitating the generation of a mammalian MSC-derived cell useful for the treatment of a neurological condition, said method comprising contacting the mesenchymal stem cells isolated in accordance with the method of aspect 54 with a stimulus to direct the differentiation of said mesenchymal stem cells to a mesenchymal phenotype.

Aspect 60. A method of therapeutically and/or prophylactically treating a neurological condition in a mammal, said method comprising administering to said mammal an effective number of mesenchymal stem cells or partially or fully differentiated MSC-derived cells, which mesenchymal stem cells have been isolated according to the method of aspect 54.

Aspect 61. An isolated population of endothelial progenitor cells or EPC-derived cells that possess ability to treat a neurological conditions, which endothelial progenitor cells have been isolated in accordance with the method of aspect 53.

Aspect 62. An isolated population of mesenchymal stem cells or MSC-derived cells, useful for treatment of a neurological condition, which mesenchymal stem cells have been isolated in accordance with the method of aspect 54.

Aspect 63. A method according to aspect 53, wherein the cellular preparation is a placenta-derived cellular population, capable of treating a neurological condition.

Aspect 64. An isolated endothelial progenitor cell that expresses at least one marker gene selected from the group consisting of MFGE8, MATN2, ELN, IGFBP2, SERPINH1, P4HA3, FN1, PKNOX2, FOXC1, NFIX, SMAD6, PRRX2, LRRC17, CTSK, PLA2G4A, DIRAS3, PDLIM3, ABCA8, CFB, PTK7, PTGFRN, SETBP1, LOC652900, SLC22A17, TANC2, SEZ6L2, ARRDC4, PODXL, MEOX2, MMP4, FAM107A, LOC647543 and SCAMP5 at a level that is at least 10% different than the expression level of a corresponding marker gene in an endothelial progenitor cell derived from umbilical cord blood (UCB), having ability to treat a neurological condition.

Aspect 65. An isolated endothelial progenitor cell according to aspect 64, which is derived from placenta.

Aspect 66. An isolated endothelial progenitor cell according to aspect 64, which expresses a CD31.sup.lo phenotypic profile.

Aspect 67. An isolated endothelial progenitor cell according to aspect 64, which further expresses at least one phenotypic profile selected from the group consisting of a CD105.sup.+ phenotypic profile, a CD144.sup.+ phenotypic profile, a CD146.sup.+ phenotypic profile, a VEGFR2.sup.+ phenotypic profile, a HLA-ABC.sup.+ phenotypic profile, a CD73.sup.− phenotypic profile and a HLA-DR.sup.− phenotypic profile.

Aspect 68. A method of treating a neurodegenerative condition comprising the steps of: a) obtaining a placentally derived regeneratve cell; b) administering said placentally derived regenerative cell at a concentration and/or frequency sufficient to induce a neuroregenerative and/or neuroprotective effect.

Aspect 69. The method of aspect 68, wherein said neurodegenerative condition is depression.

Aspect 70. The method of aspect 68, wherein said neurodegenerative condition is traumatic brain injury.

Aspect 71. The method of aspect 68, wherein said neurodegenerative condition is chronic traumatic encephalopathy.

Aspect 72. The method of aspect 68, wherein said neurodegenerative condition is Parkinson's Disease.

Aspect 73. The method of aspect 68, wherein said neurodegenerative condition is Alzheimer's Disease.

Aspect 74. The method of aspect 68, wherein said neurodegenerative condition is Minimal cognitive impairment associated with Alzheimer's Disease.

Aspect 75. The method of aspect 68, wherein said neurodegenerative condition is Post Traumatic Stress Disorder.

Aspect 76. The method of aspect 68, wherein said neurodegenerative condition is drug addiction.

Aspect 77. The method of aspect 68, wherein said neurodegenerative condition is cocaine associated neuronal damage.

Aspect 78. The method of aspect 68, wherein said neurodegenerative condition is alcohol associated neuronal damage.

Aspect 79. The method of aspect 68, wherein said neurodegenerative condition is multiple sclerosis.

Aspect 80. The method of aspect 68, wherein said neurodegenerative condition is ALS.

Aspect 81. The method of aspect 68, wherein said neurodegenerative condition is stroke.

Aspect 82. The method of aspect 68, wherein said regenerative cell derived from placental tissue is a mesenchymal stem cell.

Aspect 83. The method of aspect 68, wherein said mesenchymal stem cell is a mesenchymal progenitor cell.

Aspect 84. The method of aspect 68, wherein said mesenchymal progenitor cell expresses markers selected from a group comprising of; a) NANOG; b) OCT-4; c) SSEA-4; and d) stem cell factor receptor.

Aspect 85. The method of aspect 68, wherein said mesenchymal stem cell is isolated by a method comprising the steps of: (i) isolating a mammalian cellular population; (ii) enriching for a subpopulation of the cells of step (i), which subpopulation expresses a CD45.sup.− phenotypic profile; and (a) enriching for a subpopulation of the CD45.sup.− cells derived from step (ii) which express a CD34.sup.+ phenotypic profile and isolating the subpopulation of said CD34.sup.+ cells which express a CD31.sup.− phenotypic profile and/or (b) isolating the subpopulation of CD45.sup.− cells derived from step (ii) which express a CD34.sup.− phenotypic profile, to thereby isolate the mesenchymal stem cells.

Aspect 86. The method of aspect 68, wherein said mesenchymal stem cells are isolated by a method comprising the sequential steps of: (i) isolating a mammalian cellular population; (ii) enriching for a subpopulation of the cells of step (i), which subpopulation expresses a CD45.sup.− phenotypic profile; and (a) enriching for a subpopulation of the CD45.sup.− cells derived from step (ii) which express a CD34.sup.+ phenotypic profile and isolating the subpopulation of said CD34.sup.+ cells which express a CD31.sup.− phenotypic profile; and/or (b) isolating the subpopulation of CD45.sup.− cells derived from step (ii) which express a CD34.sup.− phenotypic profile, to thereby isolate the mesenchymal stem cells.

Aspect 87. The method according aspect 85, wherein the CD31.sup.− population is a fetal CD31.sup.− population and said mesenchymal stem cells are fetal mesenchymal stem cells.

Aspect 88. The method according to aspect 86, wherein the CD45.sup.−/CD34.sup.− mesenchymal stem cells are maternal stem cells.

Aspect 89. The method of aspect 1, wherein said regenerative cells are selected from a group of cells comprising: a) type 2 monocytes; b) reprogrammed cells; c) hematopoietic stem cells; d) mesenchymal stem cells; e) endothelial progenitor cells; f) very small embryonic-like cells and g) a stem cell.

Aspect 90. The method of aspect 89, wherein said type 2 monocytes are characterized by expression of the enzyme arginase.

Aspect 91. The method of aspect 89, wherein said type 2 monocytes are generated by exposing monocytes to a type 2 cytokine.

Aspect 92. The method of aspect 89, wherein said type 2 cytokines are selected from a group comprising of: a) IL4; b) IL-10; c) TGF-beta; and e) VEGF.

Aspect 93. The method of aspect 89, wherein said type 2 monocytes are generated by exposure to Substance P.

Aspect 94. The method of aspect 89, wherein said type 2 monocytes are derived from stromal vascular fraction of adipose tissue.

Aspect 95. The method of aspect 89, wherein said reprogrammed cells are selected from a group comprising of: a) therapeutic cells exposed to cytoplasm of a cell possessing a more immature phenotype than said target cell; b) cells are induced to dedifferentiate through the administration of a chemical agent; c) cells that are induced to dedifferentiate through transfection with genes capable of inducing dedifferentiation; and d) cells that are created as a result of a fusion with a more undifferentiated cell.

Aspect 96. The method of aspect 95, wherein said therapeutic cells exposed to cytoplasm of a cell possessing a more immature phenotype than said target cell are fibroblasts transfected with cytoplasm from a group of cells comprising of: a) embryonic stem cells; b) inducible pluripotent cells; and e) fetal stem cells.

Aspect 97. The method of aspect 96, wherein said therapeutic cells are further treated with an agent selected from: a) a histone deacetylase inhibitor; and b) a DNA methyltransferase inhibitor.

Aspect 98. The method of aspect 95, wherein said reprogrammed cell is an induced pluripotent stem cell.

Aspect 99. The method of aspect 98, wherein said induced pluripotent cells is a cell transfected with genes selected from a group comprising of: OCT4, SOX2, NANOG, and KLF-4.

Aspect 100. The method of aspect 97, wherein said chemical agent capable of inducing dedifferentiation is selected from a group of agents comprising of: valproic acid, 5-azacytidine, and trichostatin A.

Aspect 101. The method of aspect 95 wherein said reprogrammed cell is generated through fusing a fibroblast with an embryonic stem cell.

Aspect 102. The method of aspect 95, wherein said reprogrammed cell is generated through fusing a fibroblast with a parthenogenic stem cell.

Aspect 103. The method of aspect 89, wherein said hematopoietic stem cells are cells expressing markers selected from a group comprising of: a) CD34; b) CD117; c) aldehyde dehydrogenase; d) CD45; and e) CD133

Aspect 104. The method of aspect 103, wherein said hematopoietic stem cells do not express substantial levels of markers selected from a group comprising of: a) CD14; b) CD38; and c) CD56.

Aspect 105. The method of aspect 89, wherein said hematopoietic stem cells are capable of causing formation of cells derived from the myeloid, lymphoid and erythroid lineage.

Aspect 106. The method of aspect 89, wherein said mesenchymal stem cells are characterized by expression of markers selected from a group comprising of: CD90, CD105, CD73, and Stro-1.

Aspect 107. The method of aspect 106, wherein said mesenchymal stem cells lack significant expression of the markers CD14, CD34, and CD45.

Aspect 108. The method of aspect 89, wherein said endothelial progenitor cells are characterized by an agent capable of bind a molecule selected from the group of: a) CD34; b) CD133; c) KDR-1; and d) CD166.

Aspect 109. The method of aspect 108, wherein said endothelial progenitor cells are capable of forming endothelial colonies when plated in a methylcellulose culture dish.

Aspect 110. The method of aspect 89, wherein said very small embryonic like cells are less than 7 microns in diameter.

Aspect 111. The method of aspect 110, wherein said cells express a molecule selected from a group comprising of: a) wnt-5; b) CD34; c) CD133; d) Oct-4; e) Nanog; and f) SSEA-1.

Aspect 112. The method of aspect 89, wherein said stem cells are selected from a group comprising of: embryonic stem cells, cord blood stem cells, placental stem cells, bone marrow stem cells, amniotic fluid stem cells, neuronal stem cells, circulating peripheral blood stem cells, germinal stem cells, adipose tissue derived stem cells, exfoliated teeth derived stem cells, hair follicle stem cells, amnionic membrane stem cells, dermal stem cells, parthenogenically derived stem cells, reprogrammed stem cells and side population stem cells.

Aspect 113. The method of aspect 112, wherein said embryonic stem cells are totipotent.

Aspect 114. The method of aspect 112, wherein said embryonic stem cells express one or more antigens selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

Aspect 115. The method of aspect 112, wherein said cord blood stem cells are multipotent and capable of differentiating into endothelial, smooth muscle, and neuronal cells.

Aspect 116. The method of aspect 112, wherein said cord blood stem cells are identified based on expression of one or more antigens selected from a group comprising: SSEA-3, SSEA-4, CD9, CD34, c-kit, OCT-4, Nanog, and CXCR-4.

Aspect 117. The method of aspect 115, wherein said cord blood stem cells do not express one or more markers selected from a group comprising of: CD3, CD34, CD45, and CD11b.

Aspect 118. The method of aspect 112, wherein said placental stem cells are isolated from the placental structure.

Aspect 119. The method of aspect 112, wherein said placental stem cells are identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2.

Aspect 120. The method of aspect 112, wherein said bone marrow stem cells comprise of bone marrow mononuclear cells.

Aspect 121. The method of aspect 112, wherein said bone marrow stem cells are enriched for expression of CD133.

Aspect 122. The method of aspect 112, wherein said amniotic fluid stem cells are isolated by introduction of a fluid extraction means into the amniotic cavity under ultrasound guidance.

Aspect 123. The method of aspect 122, wherein said amniotic fluid stem cells are selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1.

Aspect 124. The method of aspect 122, wherein said amniotic fluid stem cells are selected based on lack of expression of one or more of the following antigens: CD34, CD45, and HLA Class II.

Aspect 125. The method of aspect 112, wherein said neuronal stem cells are selected based on expression of one or more of the following antigens: RC-2, 3CB2, BLB, Sox-2hh, GLAST, Pax 6, nestin, Muashi-1, NCAM, A2B5 and prominin.

Aspect 126. The method of aspect 112, wherein said circulating peripheral blood stem cells are characterized by ability to proliferate in vitro for a period of over 3 months.

Aspect 127. The method of aspect 112, wherein said circulating peripheral blood stem cells are characterized by expression of CD34, CXCR4, CD117, CD113, and c-met.

Aspect 128. The method of aspect 127, wherein said circulating peripheral blood stem cells lack substantial expression of differentiation associated markers.

Aspect 129. The method of aspect 128, wherein said differentiation associated markers are selected from a group comprising of CD2, CD3, CD4, CD11, CD11a, Mac-1, CD14, CD16, CD19, CD24, CD33, CD36, CD38, CD45, CD56, CD64, CD68, CD86, CD66b, and HLA-DR.

Aspect 130. The method of aspect 112, wherein said mesenchymal stem cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Aspect 131. The method of aspect 130, wherein said mesenchymal stem cells do not express substantial levels of HLA-DR, CD117, and CD45.

Aspect 132. The method of aspect 112, wherein said germinal stem cells express markers selected from a group comprising of: Oct4, Nanog, Dppa5Rbm, cyclin A2, Tex18, Stra8, Dazl, beta1-and alpha6-integrins, Vasa, Fragilis, Nobox, c-Kit, Sca-1 and Rex1.

Aspect 133. The method of aspect 112, wherein said adipose tissue derived stem cells express markers selected from a group comprising of: CD13, CD29, CD44, CD63, CD73, CD90, CD166, Aldehyde dehydrogenase (ALDH), and ABCG2.

Aspect 134. The method of aspect 112, wherein said adipose tissue derived stem cells are a population of purified mononuclear cells extracted from adipose tissue capable of proliferating in culture for more than 1 month.

Aspect 135. The method of aspect 112, wherein said exfoliated teeth derived stem cells express markers selected from a group comprising of: STRO-1, CD146 (MUC18), alkaline phosphatase, MEPE, and bFGF.

Aspect 136. The method of aspect 112, wherein said hair follicle stem cells express markers selected from a group comprising of: cytokeratin 15, Nanog, and Oct-4.

Aspect 137. The method of aspect 112, wherein said hair follicle stem cells are capable of proliferating in culture for a period of at least one month.

Aspect 138. The method of aspect 112, wherein said hair follicle stem cells secrete one or more of the following proteins when grown in culture: basic fibroblast growth factor (bFGF), endothelin-1 (ET-1) and stem cell factor (SCF).

Aspect 139. The method of aspect 112, wherein said dermal stem cells express markers selected from a group comprising of: CD44, CD13, CD29, CD90, and CD105.

Aspect 140. The method of aspect 112, wherein said dermal stem cells are capable of proliferating in culture for a period of at least one month.

Aspect 141. The method of aspect 112, wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

Aspect 142. The method of aspect 112, wherein said reprogrammed stem cells are selected from a group comprising of: cells subsequent to a nuclear transfer, cells subsequent to a cytoplasmic transfer, cells treated with a DNA methyltransferase inhibitor, cells treated with a histone deacetylase inhibitor, cells treated with a GSK-3 inhibitor, cells induced to dedifferentiate by alteration of extracellular conditions, and cells treated with various combination of the mentioned treatment conditions.

Aspect 143. The method of aspect 142, wherein said nuclear transfer comprises introducing nuclear material to a cell substantially enucleated, said nuclear material deriving from a host whose genetic profile is sought to be dedifferentiated.

Aspect 144. The method of aspect 142, wherein said cytoplasmic transfer comprises introducing cytoplasm of a cell with a dedifferentiated phenotype into a cell with a differentiated phenotype, such that said cell with a differentiated phenotype substantially reverts to a dedifferentiated phenotype.

Aspect 145. The method of aspect 142, wherein said DNA demethylating agent is selected from a group comprising of: 5-azacytidine, psammaplin A, and zebularine.

Aspect 146. The method of aspect 142, wherein said histone deacetylase inhibitor is selected from a group comprising of: valproic acid, trichostatin-A, trapoxin A and depsipeptide.

Aspect 147. The side population cells of aspect 112, wherein said cells are identified based on expression multidrug resistance transport protein (ABCG2) or ability to efflux intracellular dyes such as rhodamine-123 and or Hoechst 33342.

Aspect 148. The side population cells of aspect 147, wherein said cells are derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

Aspect 149. The method of aspect 112, wherein said committed progenitor cells are selected from a group comprising of: endothelial progenitor cells, neuronal progenitor cells, and hematopoietic progenitor cells.

Aspect 150. The method of aspect 149, wherein said committed endothelial progenitor cells are purified from the bone marrow.

Aspect 151. The method of aspect 150, wherein said committed endothelial progenitor cells are purified from peripheral blood.

Aspect 152. The method of aspect 151, wherein said committed endothelial progenitor cells are purified from peripheral blood of a patient whose committed endothelial progenitor cells are mobilized by administration of a mobilizing agent or therapy.

Aspect 153. The method of aspect 151, wherein said mobilizing agent is selected from a group comprising of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA)reductase inhibitors and small molecule antagonists of SDF-1.

Aspect 154. The method of aspect 152, wherein said mobilization therapy is selected from a group comprising of: exercise, hyperbaric oxygen, autohemotherapy by ex vivo ozonation of peripheral blood, and induction of SDF-1 secretion in an anatomical area outside of the bone marrow.

Aspect 155. The method of aspect 154, wherein said committed endothelial progenitor cells express markers selected from a group comprising of: CD31, CD34, AC133, CD146 and flk1.

Aspect 156. The method of aspect 155, wherein said committed hematopoietic cells are purified from the bone marrow.

Aspect 157. The method of aspect 149, wherein said committed hematopoietic progenitor cells are purified from peripheral blood.

Aspect 158. The method of aspect 157, wherein said committed hematopoietic progenitor cells are purified from peripheral blood of a patient whose committed hematopoietic progenitor cells are mobilized by administration of a mobilizing agent or therapy.

Aspect 159. The method of aspect 158, wherein said mobilizing agent is selected from a group comprising of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA)reductase inhibitors and small molecule antagonists of SDF-1.

Aspect 160. The method of aspect 159, wherein said mobilization therapy is selected from a group comprising of: exercise, hyperbaric oxygen, autohemotherapy by ex vivo ozonation of peripheral blood, and induction of SDF-1 secretion in an anatomical area outside of the bone marrow.

Aspect 161. The method of aspect 112, wherein said committed hematopoietic progenitor cells express the marker CD133.

Aspect 162. The method of aspect 112, wherein said committed hematopoietic progenitor cells express the marker CD34.

Aspect 163. The method of aspects 1, wherein an antioxidant is administered at a therapeutically sufficient concentration to a patient in need thereof.

Aspect 164. The method of aspect 163, wherein said antioxidant is selected from a group comprising of: ascorbic acid and derivatives thereof, alpha tocopherol and derivatives thereof, rutin, quercetin, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol.

Aspect 165. The method of aspect 112, wherein said amnionic membrane stem cell is pluripotent.

Aspect 166. The method of aspect 165, wherein said amnionic membrane stem cell possesses properties of pluripotency.

Aspect 167. The method of aspect 166, wherein said amnionic membrane stem cells are prepared by the steps of: a) separating an amniotic membrane tissue sample from chorion of a mammalian embryo; b) culturing the amniotic membrane tissue sample in culture media without any enzymes or reagents to digest the amniotic membrane tissue sample; c) preparing a single-cell culture of adherent amnionic membrane stem cells isolated from the amniotic membrane tissue sample; and d) culturing said amnionic membrane stem cells; and e) obtaining or isolating the amnionic membrane stem cells.

Aspect 168. The method of aspect 167, wherein said amniotic membrane tissue sample is washed and fragmented after step “a” and before step “b”.

Aspect 169. The method of aspect 167, wherein the amniotic membrane tissue sample is cultured in Dulbecco's modified Eagle's medium (DMEM) supplemented with 20% fetal bovine serum (FBS).

Aspect 170. The method of aspect 167 wherein said amnion-derived stem cell is positive for CD90 and CD29.

Aspect 171. The method of aspect 167 wherein said amnion-derived stem cell is negative for CD45 and CD11b.

Aspect 172. The method of aspect 167 wherein said amnion-derived stem cell is capable of differentiation into osteoblasts and adipocytes.

### REFERENCES

1. Liauw, S. L., P. P. Connell, and R. R. Weichselbaum, New paradigms and future challenges in radiation oncology: an update of biological targets and technology. Sci Transl Med, 2013. 5(173): p. 173sr2.

2. Jannoun, L. and H. J. Bloom, Long-term psychological effects in children treated for intracranial tumors. Int J Radiat Oncol Biol Phys, 1990. 18(4): p. 747-53.

3. Peissner, W., et al., Ionizing radiation-induced apoptosis of proliferating stem cells in the dentate gyrus of the adult rat hippocampus. Brain Res Mol Brain Res, 1999. 71(1): p. 61-8.

4. Pereira Dias, G., et al., Consequences of cancer treatments on adult hippocampal neurogenesis: implications for cognitive function and depressive symptoms. Neuro Oncol, 2014. 16(4): p. 476-92.

5. Mizumatsu, S., et al., Extreme sensitivity of adult neurogenesis to low doses of X-irradiation. Cancer Res, 2003. 63(14): p. 4021-7.

6. Abayomi, O. K., Pathogenesis of cognitive decline following therapeutic irradiation for head and neck tumors. Acta Oncol, 2002. 41(4): p. 346-51.

7. Roman, D. D. and P. W. Sperduto, Neuropsychological effects of cranial radiation: current knowledge and future directions. Int J Radiat Oncol Biol Phys, 1995. 31(4): p. 983-98.

8. Ramanan, S., et al., The PPARalpha agonist fenofibrate preserves hippocampal neurogenesis and inhibits microglial activation after whole-brain irradiation. Int J Radiat Oncol Biol Phys, 2009. 75(3): p. 870-7.

9. Obeng, E. A., et al., Proteasome inhibitors induce a terminal unfolded protein response in multiple myeloma cells. Blood, 2006. 107(12): p. 4907-16.

10. Zhang, X. Y., et al., Lentiviral vectors for sustained transgene expression in human bone marrow-derived stromal cells. Mol Ther, 2002. 5(5 Pt 1): p. 555-65.

11. Kyriakou, C. A., et al., Human mesenchymal stem cells (hMSCs) expressing truncated soluble vascular endothelial growth factor receptor (tsFlk-1) following Lentiviral-mediated gene transfer inhibit growth of Burkitt's lymphoma in a murine model. J Gene Med, 2006. 8(3): p. 253-64.

12. Worsham, D. N., et al., In vivo gene transfer into adult stem cells in unconditioned mice by in situ delivery of a lentiviral vector. Mol Ther, 2006. 14(4): p. 514-24.

13. Rabin, N., et al., A new xenograft model of myeloma bone disease demonstrating the efficacy of human mesenchymal stem cells expressing osteoprotegerin by lentiviral gene transfer. Leukemia, 2007. 21(10): p. 2181-91.

14. Kallifatidis, G., et al., Improved lentiviral transduction of human mesenchymal stem cells for therapeutic intervention in pancreatic cancer. Cancer Gene Ther, 2008. 15(4): p. 231-40.

15. Meyerrose, T. E., et al., Lentiviral-transduced human mesenchymal stem cells persistently express therapeutic levels of enzyme in a xenotransplantation model of human disease. Stem Cells, 2008. 26(7): p. 1713-22.

16. McGinley, L., et al., Lentiviral vector mediated modification of mesenchymal stem cells & enhanced survival in an in vitro model of ischaemia. Stem Cell Res Ther, 2011. 2(2): p. 12.

17. Liang, X., et al., Human bone marrow mesenchymal stem cells expressing SDF-1 promote hematopoietic stem cell function of human mobilised peripheral blood CD34+ cells in vivo and in vitro. Int J Radiat Biol, 2010. 86(3): p. 230-7.

18. Glavaski-Joksimovic, A., et al., Glial cell line-derived neurotrophic factor-secreting genetically modified human bone marrow-derived mesenchymal stem cells promote recovery in a rat model of Parkinson's disease. J Neurosci Res, 2010. 88(12): p. 2669-81.

19. Liu, A. M., et al., Umbilical cord-derived mesenchymal stem cells with forced expression of hepatocyte growth factor enhance remyelination and functional recovery in a rat intracerebral hemorrhage model. Neurosurgery, 2010. 67(2): p. 357-65; discussion 365-6.

20. Yu, Y. S., et al., AKT-modified autologous intracoronary mesenchymal stem cells prevent remodeling and repair in swine infarcted myocardium. Chin Med J (Engl), 2010. 123(13): p. 1702-8.

21. Mueller, L. P., et al., TRAIL-transduced multipotent mesenchymal stromal cell s(TRAIL-MSC) overcome TRAIL resistance in selected CRC cell lines in vitro and in vivo. Cancer Gene Ther, 2011. 18(4): p. 229-39.

22. Yan, C., et al., Suppression of orthotopically implanted hepatocarcinoma in mice by umbilical cord-derived mesenchymal stem cells with sTRAIL gene expression driven by AFP promoter. Biomaterials, 2014. 35(9): p. 3035-43.

23. Deng, Q., et al., TRAIL-secreting mesenchymal stem cells promote apoptosis in heat-shock-treated liver cancer cells and inhibit tumor growth in nude mice. Gene Ther, 2014. 21(3): p. 317-27.

24. Sage, E. K., et al., Systemic but not topical TRAIL-expressing mesenchymal stem cells reduce tumour growth in malignant mesothelioma. Thorax, 2014. 69(7): p. 638-47.

25. Lian, W. S., et al., In vivo therapy of myocardial infarction with mesenchymal stem cells modified with prostaglandin I synthase gene improves cardiac performance in mice. Life Sci, 2011. 88(9-10): p. 455-64.

26. Maijenburg, M. W., et al., Nuclear receptors Nur77 and Nurr1 modulate mesenchymal stromal cell migration. Stem Cells Dev, 2012. 21(2): p. 228-38.

27. Harper, M. M., et al., Transplantation of BDNF-secreting mesenchymal stem cells provides neuroprotection in chronically hypertensive rat eyes. Invest Ophthalmol Vis Sci, 2011. 52(7): p. 4506-15.

28. Zou, D., et al., In vitro study of enhanced osteogenesis induced by HIF-lalpha-transduced bone marrow stem cells. Cell Prolif, 2011. 44(3): p. 234-43.

29. Saito, S., et al., Mesenchymal stem cells stably transduced with a dominant-negative inhibitor of CCL2 greatly attenuate bleomycin-induced lung damage. Am J Pathol, 2011. 179(3): p. 1088-94.

30. Seo, K. W., et al., Anti-tumor effects of canine adipose tissue-derived mesenchymal stromal cell-based interferon-beta gene therapy and cisplatin in a mouse melanoma model. Cytotherapy, 2011. 13(8): p. 944-55.

31. Yang, H. M., et al., Enhancement of the immunosuppressive effect of human adipose tissue-derived mesenchymal stromal cells through HLA-G1 expression. Cytotherapy, 2012. 14(1): p. 70-9.

32. Liang, X. J., et al., Differentiation of human umbilical cord mesenchymal stem cells into hepatocyte-like cells by hTERT gene transfection in vitro. Cell Biol Int, 2012. 36(2): p. 215-21.

33. Fei, S., et al., The antitumor effect of mesenchymal stem cells transduced with a lentiviral vector expressing cytosine deaminase in a rat glioma model. J Cancer Res Clin Oncol, 2012. 138(2): p. 347-57.

34. Jaganathan, B. G. and D. Bonnet, Human mesenchymal stromal cells senesce with exogenous OCT4. Cytotherapy, 2012. 14(9): p. 1054-63.

35. Han, S. H., et al., Effect of ectopic OCT4 expression on canine adipose tissue-derived mesenchymal stem cell proliferation. Cell Biol Int, 2014. 38(10): p. 1163-73.

36. Shangguan, L., et al., Inhibition of TGF-beta/Smad signaling by BAMBI blocks differentiation of human mesenchymal stem cells to carcinoma-associated fibroblasts and abolishes their protumor effects. Stem Cells, 2012. 30(12): p. 2810-9.

37. Kearns-Jonker, M., et al., Genetically Engineered Mesenchymal Stem Cells Influence Gene Expression in Donor Cardiomyocytes and the Recipient Heart. J Stem Cell Res Ther, 2012. S1.

38. Ma, G. L., et al., [Study of inhibiting and killing effects of transgenic LIGHT human umbilical cord blood mesenchymal stem cells on stomach cancer]. Zhonghua Wei Chang Wal Ke Za Zhi, 2012. 15(11): p. 1178-81.

39. Huang, F., et al., Mesenchymal stem cells modified with miR-126 release angiogenic factors and activate Notch ligand Delta-like-4, enhancing ischemic angiogenesis and cell survival. Int J Mol Med, 2013. 31(2): p. 484-92.

40. Huang, F., et al., Overexpression of miR-126 promotes the differentiation of mesenchymal stem cells toward endothelial cells via activation of PI3K/Akt and MAPK/ERK pathways and release of paracrine factors. Biol Chem, 2013. 394(9): p. 1223-33.

41. Fang, Z., et al., Differentiation of GFP-Bcl-2-engineered mesenchymal stem cells towards a nucleus pulposus-like phenotype under hypoxia in vitro. Biochem Biophys Res Commun, 2013. 432(3): p. 444-50.

42. Madonna, R., et al., Transplantation of mesenchymal cells rejuvenated by the overexpression of telomerase and myocardin promotes revascularization and tissue repair in a murine model of hindlimb ischemia. Circ Res, 2013. 113(7): p. 902-14.

43. Zang, Y., et al., [Influence of CXCR4 overexpressed mesenchymal stem cells on hematopoietic recovery of irradiated mice]. Zhongguo Shi Yan Xue Ye Xue Za Zhi, 2013. 21(5): p. 1261-5.

44. Cao, Z., et al., Protective effects of mesenchymal stem cells with CXCR4 up-regulation in a rat renal transplantation model. PLoS One, 2013. 8(12): p. e82949.

45. Liu, S., et al., Overexpression of Wnt11 promotes chondrogenic differentiation of bone marrow-derived mesenchymal stem cells in synergism with TGF-beta. Mol Cell Biochem, 2014. 390(1-2): p. 123-31.

46. Zhu, Y., et al., Mesenchymal stem cell-based NK4 gene therapy in nude mice bearing gastric cancer xenografts. Drug Des Devel Ther, 2014. 8: p. 2449-62.

47. Yu, B., et al., Enhanced mesenchymal stem cell survival induced by GATA-4 overexpression is partially mediated by regulation of the miR-15 family. Int J Biochem Cell Biol, 2013. 45(12): p. 2724-35.

48. Xu, W., et al., Basic fibroblast growth factor expression is implicated in mesenchymal stem cells response to light-induced retinal injury. Cell Mol Neurobiol, 2013. 33(8): p. 1171-9.

49. Li, W., et al., Bcl-2 engineered MSCs inhibited apoptosis and improved heart function. Stem Cells, 2007. 25(8): p. 2118-27.

50. Tsubokawa, T., et al., Impact of anti-apoptotic and anti-oxidative effects of bone marrow mesenchymal stem cells with transient overexpression of heme oxygenase-1 on myocardial ischemia. Am J Physiol Heart Circ Physiol, 2010. 298(5): p. H1320-9.

51. Zhou, H., et al., Exendin-4 protects adipose-derived mesenchymal stem cells from apoptosis induced by hydrogen peroxide through the PI3K/Akt-Sfrp2pathways. Free Radic Biol Med, 2014. 77: p. 363-75.

52. Le Blanc, K., et al., Treatment of severe acute graft-versus-host disease with third party haploidentical mesenchymal stem cells. Lancet, 2004. 363(9419): p. 1439-41.

53. Lazarus, H. M., et al., Cotransplantation of HLA-identical sibling culture-expanded mesenchymal stem cells and hematopoietic stem cells in hematologic malignancy patients. Biol Blood Marrow Transplant, 2005. 11(5): p. 389-98.

54. Bernardo, M. E., et al., Optimization of in vitro expansion of human multipotent mesenchymal stromal cells for cell-therapy approaches: further insights in the search for a fetal calf serum substitute. J Cell Physiol, 2007. 211(1): p. 121-30.

55. Reinisch, A., et al., Humanized system to propagate cord blood-derived multipotent mesenchymal stromal cells for clinical application. Regen Med, 2007. 2(4): p. 371-82.

56. Capelli, C., et al., Human platelet lysate allows expansion and clinical grade production of mesenchymal stromal cells from small samples of bone marrow aspirates or marrow filter washouts. Bone Marrow Transplant, 2007. 40(8): p. 785-91.

57. Lataillade, J. J., et al., New approach to radiation burn treatment by dosimetry-guided surgery combined with autologous mesenchymal stem cell therapy. Regen Med, 2007. 2(5): p. 785-94.

58. Seshareddy, K., D. Troyer, and M. L. Weiss, Method to isolate mesenchymal-like cells from Wharton's Jelly of umbilical cord. Methods Cell Biol, 2008. 86: p. 101-19.

59. Sensebe, L., Clinical grade production of mesenchymal stem cells. Biomed Mater Eng, 2008. 18(1 Suppl): p. S3-10.

60. Sotiropoulou, P. A., S. A. Perez, and M. Papamichail, Clinical grade expansion of human bone marrow mesenchymal stem cells. Methods Mol Biol, 2007. 407: p. 245-63.

61. Shetty, P., et al., Clinical grade mesenchymal stem cells transdifferentiated under xenofree conditions alleviates motor deficiencies in a rat model of Parkinson's disease. Cell Biol Int, 2009. 33(8): p. 830-8.

62. Zhang, X., et al., Cotransplantation of HLA-identical mesenchymal stem cells and hematopoietic stem cells in Chinese patients with hematologic diseases. Int J Lab Hematol, 2010. 32(2): p. 256-64.

63. Arrigoni, E., et al., Isolation, characterization and osteogenic differentiation of adipose-derived stem cells: from small to large animal models. Cell Tissue Res, 2009. 338(3): p. 401-11.

64. Grisendi, G., et al., GMP-manufactured density gradient media for optimized mesenchymal stromal/stem cell isolation and expansion. Cytotherapy, 2010. 12(4): p. 466-77.

65. Prasad, V. K., et al., Efficacy and safety of ex vivo cultured adult human mesenchymal stem cells (Prochymal) in pediatric patients with severe refractory acute graft-versus-host disease in a compassionate use study. Biol Blood Marrow Transplant, 2011. 17(4): p. 534-41.

66. Sensebe, L., P. Bourin, and K. Tarte, Good manufacturing practices production of mesenchymal stem/stromal cells. Hum Gene Ther, 2011. 22(1): p. 19-26.

67. Capelli, C., et al., Minimally manipulated whole human umbilical cord is a rich source of clinical-grade human mesenchymal stromal cells expanded in human platelet lysate. Cytotherapy, 2011. 13(7): p. 786-801.

68. Ilic, N., et al., Manufacture of clinical grade human placenta-derived multipotent mesenchymal stromal cells. Methods Mol Biol, 2011. 698: p. 89-106.

69. Santos, F., et al., Toward a clinical-grade expansion of mesenchymal stem cells from human sources: a microcarrier-based culture system under xeno-free conditions. Tissue Eng Part C Methods, 2011. 17(12): p. 1201-10.

70. Timmins, N. E., et al., Closed system isolation and scalable expansion of human placental mesenchymal stem cells. Biotechnol Bioeng, 2012. 109(7): p. 1817-26.

71. Warnke, P. H., et al., A clinically-feasible protocol for using human platelet lysate and mesenchymal stem cells in regenerative therapies. J Craniomaxillofac Surg, 2013. 41(2): p. 153-61.

72. Fekete, N., et al., GMP-compliant isolation and large-scale expansion of bone marrow-derived MSC. PLoS One, 2012. 7(8): p. e43255.

73. Hanley, P. J., et al., Manufacturing mesenchymal stromal cells for phase I clinical trials. Cytotherapy, 2013. 15(4): p. 416-22.

74. Trojahn Kolle, S. F., et al., Pooled human platelet lysate versus fetal bovine serum-investigating the proliferation rate, chromosome stability and angiogenic potential of human adipose tissue-derived stem cells intended for clinical use. Cytotherapy, 2013. 15(9): p. 1086-97.

75. Veronesi, E., et al., Transportation conditions for prompt use of ex vivo expanded and freshly harvested clinical-grade bone marrow mesenchymal stromal/stem cells for bone regeneration. Tissue Eng Part C Methods, 2014. 20(3): p. 239-51.

76. Dolley-Sonneville, P. J., L. E. Romeo, and Z. K. Melkoumian, Synthetic surface for expansion of human mesenchymal stem cells in xeno-free, chemically defined culture conditions. PLoS One, 2013. 8(8): p. e70263.

77. Siciliano, C., et al., Optimization of the isolation and expansion method of human mediastinal-adipose tissue derived mesenchymal stem cells with virally inactivated GMP-grade platelet lysate. Cytotechnology, 2015. 67(1): p. 165-74.

78. Martins, J. P., et al., Towards an advanced therapy medicinal product based on mesenchymal stromal cells isolated from the umbilical cord tissue: quality and safety data. Stem Cell Res Ther, 2014. 5(1): p. 9.

79. Iudicone, P., et al., Pathogen-free, plasma-poor platelet lysate and expansion of human mesenchymal stem cells. J Transl Med, 2014. 12: p. 28.

80. Skrahin, A., et al., Autologous mesenchymal stromal cell infusion as adjunct treatment in patients with multidrug and extensively drug-resistant tuberculosis: an open-label phase 1 safety trial. Lancet Respir Med, 2014. 2(2): p. 108-22.

81. Ikebe, C. and K. Suzuki, Mesenchymal stem cells for regenerative therapy: optimization of cell preparation protocols. Biomed Res Int, 2014. 2014: p. 951512.

82. Chatzistamatiou, T. K., et al., Optimizing isolation culture and freezing methods to preserve Wharton's jelly's mesenchymal stem cell (MSC) properties: an MSC banking protocol validation for the Hellenic Cord Blood Bank. Transfusion, 2014. 54(12): p. 3108-20.

83. Swamynathan, P., et al., Are serum free and xeno-free culture conditions ideal for large scale clinical grade expansion of Wharton's jelly derived mesenchymal stem cells? A comparative study. Stem Cell Res Ther, 2014. 5(4): p. 88.

84. Vaes, B., et al., Culturing protocols for human multipotent adult stem cells. Methods Mol Biol, 2015. 1235: p. 49-58.

85. Devito, L., et al., Wharton's jelly mesenchymal stromal/stem cells derived under chemically defined animal product-free low oxygen conditions are rich in MSCA-1(+) subpopulation. Regen Med, 2014. 9(6): p. 723-32.

86. Kessler, R. C., et al., Posttraumatic stress disorder in the National Comorbidity Survey. Arch Gen Psychiatry, 1995. 52(12): p. 1048-60.

87. Kessler, R. C., et al., Prevalence, severity, and comorbidity of 12-month DSM-IV disorders in the National Comorbidity Survey Replication. Arch Gen Psychiatry, 2005. 62(6): p. 617-27.

88. Laufer, R. S., E. Brett, and M. S. Gallops, Dimensions of posttraumatic stress disorder among Vietnam veterans. J Nery Ment Dis, 1985. 173(9): p. 538-45.

89. Kang, H. K., et al., Post-traumatic stress disorder and chronic fatigue syndrome-like illness among Gulf War veterans: a population-based survey of 30,000 veterans. Am J Epidemiol, 2003. 157(2): p. 141-8.

90. Seal, K. H., et al., Bringing the war back home: mental health disorders among 103,788 US veterans returning from Iraq and Afghanistan seen at Department of Veterans Affairs facilities. Arch Intern Med, 2007. 167(5): p. 476-82.

91. Friedman, M. J., Finalizing PTSD in DSM-5: getting here from there and where to go next. J Trauma Stress, 2013. 26(5): p. 548-56.

92. Schell T L, M.G.S.o.i.p.d.f.O.O.I.T.T., Jaycox L H, editors. Invisible Wounds of war: psychological and cognitive injuries, their consequences, and services to assist recovery. Santa Monica, Calif.: Rand Corporation; 2008. pp. 87-115.

93. Kessler, R. C., Posttraumatic stress disorder: the burden to the individual and to society. J Clin Psychiatry, 2000. 61 Suppl 5: p. 4-12; discussion 13-4.

94. Zatzick, D. F., et al., Posttraumatic stress disorder and functioning and quality of life outcomes in a nationally representative sample of male Vietnam veterans. Am J Psychiatry, 1997. 154(12): p. 1690-5.

95. Koenen, K. C., et al., Persisting posttraumatic stress disorder symptoms and their relationship to functioning in Vietnam veterans: a 14-year follow-up. J Trauma Stress, 2008. 21(1): p. 49-57.

96. Schnurr, P. P., et al., Posttraumatic stress disorder and quality of life: extension of findings to veterans of the wars in Iraq and Afghanistan. Clin Psychol Rev, 2009. 29(8): p. 727-35.

97. Breslau, N., et al., A second look at comorbidity in victims of trauma: the posttraumatic stress disorder-major depression connection. Biol Psychiatry, 2000. 48(9): p. 902-9.

98. Braakman, M. H., F. A. Kortmann, and W. van den Brink, Validity of ′post-traumatic stress disorder with secondary psychotic features': a review of the evidence. Acta Psychiatr Scand, 2009. 119(1): p. 15-24.

99. Rona, R. J., et al., The impact of posttraumatic stress disorder on impairment in the UK military at the time of the Iraq war. J Psychiatr Res, 2009. 43(6): p. 649-55.

100. Friedman, M. J., Posttraumatic stress disorder among military returnees from Afghanistan and Iraq. Am J Psychiatry, 2006. 163(4): p. 586-93.

101. Hoge, C. W., J. L. Auchterlonie, and C. S. Milliken, Mental health problems, use of mental health services, and attrition from military service after returning from deployment to Iraq or Afghanistan. JAMA, 2006. 295(9): p. 1023-32.

102. Mills, K. L., et al., Trauma, PTSD, and substance use disorders: findings from the Australian National Survey of Mental Health and Well-Being. Am J Psychiatry, 2006. 163(4): p. 652-8.

103. Cohen, B. E., et al., Posttraumatic stress disorder and health-related quality of life in patients with coronary heart disease: findings from the Heart and Soul Study. Arch Gen Psychiatry, 2009. 66(11): p. 1214-20.

104. Kang, H. K. and T. A. Bullman, Risk of suicide among US veterans after returning from the Iraq or Afghanistan war zones. JAMA, 2008. 300(6): p. 652-3.

105. Johnson, D. R., et al., Long-term course of treatment-seeking Vietnam veterans with posttraumatic stress disorder: mortality, clinical condition, and life satisfaction. J Nery Ment Dis, 2004. 192(1): p. 35-41.

106. Outcalt, S. D., et al., Health care utilization among veterans with pain and posttraumatic stress symptoms. Pain Med, 2014. 15(11): p. 1872-9.

107. Bernardy, N. C., et al., Prescribing trends in veterans with posttraumatic stress disorder. J Clin Psychiatry, 2012. 73(3): p. 297-303.

108. Brady, K., et al., Efficacy and safety of sertraline treatment of posttraumatic stress disorder: a randomized controlled trial. JAMA, 2000. 283(14): p. 1837-44.

109. Davidson, J. R., et al., Multicenter, double-blind comparison of sertraline and placebo in the treatment of posttraumatic stress disorder. Arch Gen Psychiatry, 2001. 58(5): p. 485-92.

110. Marshall, R. D., et al., Efficacy and safety of paroxetine treatment for chronic PTSD: a fixed-dose, placebo-controlled study. Am J Psychiatry, 2001. 158(12): p. 1982-8.

111. Tucker, P., et al., Paroxetine in the treatment of chronic posttraumatic stress disorder: results of a placebo-controlled, flexible-dosage trial. J Clin Psychiatry, 2001. 62(11): p. 860-8.

112. Hertzberg, M. A., et al., Lack of efficacy for fluoxetine in PTSD: a placebo controlled trial in combat veterans. Ann Clin Psychiatry, 2000. 12(2): p. 101-5.

113. Friedman, M. J., et al., Randomized, double-blind comparison of sertraline and placebo for posttraumatic stress disorder in a Department of Veterans Affairs setting. J Clin Psychiatry, 2007. 68(5): p. 711-20.

114. Ursano, R. J., et al., Practice guideline for the treatment of patients with acute stress disorder and posttraumatic stress disorder. Am J Psychiatry, 2004. 161(11 Suppl): p. 3-31.

115. Davidson, J., et al., Treatment of posttraumatic stress disorder with venlafaxine extended release: a 6-month randomized controlled trial. Arch Gen Psychiatry, 2006. 63(10): p. 1158-65.

116. Davidson, J., et al., Venlafaxine extended release in posttraumatic stress disorder: a sertraline-and placebo-controlled study. J Clin Psychopharmacol, 2006. 26(3): p. 259-67.

117. Raskind, M. A., et al., A trial of prazosin for combat trauma PTSD with nightmares in active-duty soldiers returned from Iraq and Afghanistan. Am J Psychiatry, 2013. 170(9): p. 1003-10.

118. www.healthquality.va.goviguidelines/MH/ptsd/cpg_PTSD-FULL-201011612.pdf.

119. Foa, E. B. and M. J. Kozak, Emotional processing of fear: exposure to corrective information. Psychol Bull, 1986. 99(1): p. 20-35.

120. Difede, J., et al., D-cycloserine augmentation of exposure therapy for pos-traumatic stress disorder: a pilot randomized clinical trial. Neuropsychopharmacology, 2014. 39(5): p. 1052-8.

121. Kim, J., et al., Amygdala depotentiation and fear extinction. Proc Natl Acad Sci USA, 2007. 104(52): p. 20955-60.

122. Hong, I., et al., Extinction of cued fear memory involves a distinct form of depotentiation at cortical input synapses onto the lateral amygdala. Eur J Neurosci, 2009. 30(11): p. 2089-99.

123. Foa E B, K. T., Friedman M. (eds). (2000 Effective Treatments for PTSD: Practice guidelines from the International Society for Traumatic Stress Studies Guilford Press: New York, N.Y., USA.

124. Misanin, J. R., R. R. Miller, and D. J. Lewis, Retrograde amnesia produced by electroconvulsive shock after reactivation of a consolidated memory trace. Science, 1968. 160(3827): p. 554-5.

125. Flood, J. F., et al., Effects of protein synthesis inhibition on memory for active avoidance training. Physiol Behav, 1975. 14(2): p. 177-84.

126. Flood, J. F., et al., Relation of memory formation to controlled amounts of brain protein synthesis. Physiol Behav, 1975. 15(1): p. 97-102.

127. Nader, K., G. E. Schafe, and J. E. Le Doux, Fear memories require protein synthesis in the amygdala for reconsolidation after retrieval. Nature, 2000. 406(6797): p. 722-6.

128. Hirsch, S. J., et al., CA1-specific deletion of NMDA receptors induces abnormal renewal of a learned fear response. Hippocampus, 2015. 25(11): p. 1374-9.

129. Vieira, P. A., et al., Prefrontal NMDA receptors expressed in excitatory neurons control fear discrimination and fear extinction. Neurobiol Learn Mem, 2015. 119: p. 52-62.

130. Furini, C., J. Myskiw, and I. Izquierdo, The learning of fear extinction. Neurosci Biobehav Rev, 2014. 47: p. 670-83.

131. Morrison, F. G. and K. J. Ressler, From the neurobiology of extinction to improved clinical treatments. Depress Anxiety, 2014. 31(4): p. 279-90.

132. Zimmerman, J. M. and S. Maren, NMDA receptor antagonism in the basolateral but not central amygdala blocks the extinction of Pavlovian fear conditioning in rats. Eur J Neurosci, 2010. 31(9): p. 1664-70.

133. Ledgerwood, L., R. Richardson, and J. Cranney, Effects of D-cycloserine on extinction of conditioned freezing. Behav Neurosci, 2003. 117(2): p. 341-9.

1. A method of protecting non-neoplastic cells from cellular damaging effects of a brain cancer directed therapy, said method comprising the steps of: a) obtaining a cell with regenerative potential; and b) administering said cell in a manner to allow chemotaxis and/or proximity to non-malignant brain tissue at a concentration and frequency sufficient to provide selective protection of non-malignant tissue from effects of chemotherapy and/or radiation therapy.

2. The method of claim 1, wherein said brain cancer directed therapy comprises therapies selected from a group comprising of: a) radiation therapy; b) chemotherapy; c) surgery; d) metabolic therapy; and e) immunotherapy.

3. The method of claim 1, wherein said cell with regenerative potential is a mesenchymal stem cell.

4. The method of claim 3, wherein said mesenchymal stem cell expresses proteins selected from a group comprising of ; a) CD73; b) CD90; and c) CD105

5. The method of claim 4, wherein said mesenchymal stem cells possess ability to secrete cytokines selected from a group comprising of; a) IFN-gamma; b) TNF-alpha; c) IL-2; d) IL-7; e) IL-12; f) IL-15; g) IL-17; h) IL-18; i) IL-21; j) IL-23; k) IL-27; 1) IL-33; m) HMGB-1; and n) TRAIL.

6. The method of claim 4, wherein said mesenchymal stem cell is modified to express an angiogenic polypeptide selected from a group comprising of: activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, collagen receptors .alpha..sub. 1.beta..sub. 1 and .alpha..sub. 2.beta..sub. 1, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin and fibronectin receptor .alpha.5.beta. 1, Factor X, HB-EGF, HBNF, HGF, HUAF, heart derived inhibitor of vascular cell proliferation, Ill, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-.beta., PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-.beta., and TGF-.beta. receptors, TIMPs, TNF-alphatransferrin, thrombospondin, urokinase, VEGF-A, VEGF-B, VEGF-C, VEGF-D, VEGF-E, VEGF, VEGF.sub.164, VEGI, EG-VEGF.

7. A method of treating a neurodegenerative condition comprising the steps of: a) obtaining a placentally derived regenerative cell; b) administering said placentally derived regenerative cell at a concentration and/or frequency sufficient to induce a neuroregenerative and/or neuroprotective effect.

8. The method of claim 7, wherein said neurodegenerative condition is depression.

9. The method of claim 7, wherein said neurodegenerative condition is traumatic brain injury.

10. The method of claim 7, wherein said neurodegenerative condition is chronic traumatic encephalopathy.

11. The method of claim 7, wherein said neurodegenerative condition is Parkinson's Disease.

12. The method of claim 7, wherein said neurodegenerative condition is Alzheimer's Disease.

13. The method of claim 7, wherein said neurodegenerative condition is Minimal cognitive impairment associated with Alzheimer's Disease.

14. The method of claim 7, wherein said neurodegenerative condition is Post Traumatic Stress Disorder.

15. The method of claim 7, wherein said neurodegenerative condition is drug addiction.

16. The method of claim 7, wherein said neurodegenerative condition is stroke.

17. The method of claim 7, wherein said regenerative cell derived from placental tissue is a mesenchymal stem cell.

18. The method of claim 7, wherein said mesenchymal progenitor cell expresses markers selected from a group comprising of; a) NANOG; b) OCT-4; c) SSEA-4; and d) stem cell factor receptor.

19. The method of claim 7, wherein said mesenchymal stem cell is isolated by a method comprising the steps of: (i) isolating a mammalian cellular population; (ii) enriching for a subpopulation of the cells of step (i), which subpopulation expresses a CD45.sup.− phenotypic profile; and (a) enriching for a subpopulation of the CD45.sup.− cells derived from step (ii) which express a CD34.sup.+ phenotypic profile and isolating the subpopulation of said CD34.sup.+ cells which express a CD31.sup.− phenotypic profile and/or (b) isolating the subpopulation of CD45.sup.− cells derived from step (ii) which express a CD34.sup.− phenotypic profile, to thereby isolate the mesenchymal stem cells.
